(19) United States
(12) Patent Application Publication
(10) Pub. No.: US 2024/0180893 A1
(43) Pub. Date: Jun. 6, 2024

(54) METHODS OF TREATMENT OF BREAST CANCER

(71) Applicant: AstraZeneca AB, Södertälje (SE)

(72) Inventors: Justin Pieter Oliver LINDEMANN, Cambridge (GB); Teresa Caroline Maria Klinowska, Cambridge (GB); Susan Mary GALBRAITH, Cambridge (GB); Christopher Jon MORROW, Cambridge (GB)

(21) Appl. No.: 18/492,216

(22) Filed: Oct. 23, 2023

Related U.S. Application Data
(60) Provisional application No. 63/384,170, filed on Nov. 17, 2022.

(51) Int. Cl. A61K 31/4745; A61K 9/00; A61P 35/00; A61P 35/04; C12Q 1/6886

(52) U.S. Cl. CPC A61K 31/4745; A61K 9/0053; A61P 35/00; A61P 35/04; C12Q 1/6886; C12Q 2600/156

(57) ABSTRACT

The present specification relates methods of treatment of hormone receptor positive (HR+), human epidermal growth factor receptor 2 negative (HER2−), metastatic or loco-regionally recurrent breast cancer, comprising administering a next generation selective estrogen receptor degrader (ngSERD), for example camizestrant, to a patient suffering from such cancer, characterised in that the cancer has recurred or progressed following at least one prior line of endocrine therapy.

### FIELD

The present specification relates to methods of treatment of hormone receptor positive (HR+), human epidermal growth factor receptor 2 negative (HER2−), metastatic or loco-regionally recurrent breast cancer, comprising administering a next generation selective estrogen receptor degrader (ngSERD) to a patient suffering from such cancer, characterised in that the cancer has recurred or progressed following at least one prior line of endocrine therapy. The specification also relates to the use of a ngSERD for use in the treatment of such breast cancers and the use of a ngSERD for the manufacture of a medicament for the treatment of such breast cancers.

### BACKGROUND

Breast cancer is the most frequently diagnosed malignancy in women globally and is the leading cause of cancer mortality in women worldwide. Locally advanced (inoperable) and/or metastatic breast cancer (MBC) remains essentially incurable. Recent advances in MBC therapy indicate that the concept of MBC as a chronic disease controlled by sequential therapies over a long period is realistic, at least for certain subgroups (see e.g. Harbeck and Gnant, Lancet 2017; 389(10074):1134-50). As such, the treatment goal is to prolong progression free survival (PFS) and overall survival (OS) while maintaining quality of life.

Hormone receptor-positive (HR+), human epidermal growth factor receptor 2 negative (HER2−) tumours account for more than two-thirds of all breast cancers (see https://seer.cancer.gov/statfacts/html/breast-subtypes.html, accessed Oct. 31, 2022). Endocrine therapy (ET) has been a backbone of care for HR+ breast cancer for decades, albeit the standard of care, use in monotherapy or in combination, and preferred ET has evolved over time. One class of ET, selective estrogen receptor modulators (SERMs), bind to estrogen receptors (ER) thereby preventing breast tumour growth stimulus otherwise provided by binding of endogenous estrogens to the ER. Examples of SERMs approved by the FDA for treatment of breast cancer include tamoxifen and toremifene. A second class of ET, aromatase inhibitors (Ais), block the activity of the aromatase enzyme, and in so doing block estrogen biosynthesis. Al therapy thus prevents the estrogen mediated activation of the ER, albeit instead of directly blocking the ligand/receptor interaction as is the case with SERMs, Ais deplete the pool of ligand available for receptor activation. Examples of Ais approved by the FDA for treatment of breast cancer include anastrazole and letrazole. Ais are principally used in post-menopausal woman albeit they can be used in the premenopausal context if combined with a suppressor of ovarian activity such as goserelin or leuprolide. A third class of ET are selective estrogen receptor degraders (SERDs). Fulvestrant (e.g. Faslodex®) is the only SERD presently approved for clinical use. Although like SERMs fulvestrant does bind to the estrogen receptor, it does not mimic estrogen and is therefore referred to as a pure antiestrogen. In addition, binding of fulvestrant to the ER causes degradation of the estrogen receptor. The clinical utility of fulvestrant has become better understood in recent years and this has led to its use in earlier lines of therapy. One factor that perhaps slowed initial uptake of fulvestrant in the clinic is the fact that it is administered as an intramuscular injection on a once monthly basis. This fact, and the realisation that more frequent dosing with an oral next generation SERD may offer greater net degradation of the estrogen receptor over time, has led to a great deal of interest in the development of oral next generation SERDs.

Mutation of the estrogen receptor (ESR1m) affecting the ligand binding domain of ERα results in constitutively active, estrogen independent ER signaling, that negates the impact of AI and SERMs. Clinically, ESR1m is associated with acquired resistance to AI, most commonly occurring in patients who have been treated with an AI in the advanced breast cancer setting. Today, in many countries the majority of advanced breast cancer patients receive AI in the form of combination with a CDK4/6i, rather than as monotherapy, and translational research from PALOMA-3 and MONARCH-3 have shown ESR1m is frequently acquired in patients treated with CDK4/6 inhibitor+ET (see e.g. Goetz et al. J. Clin. Oncol. 2020; 38 (Suppl 15):3519; O'Leary et al. Cancer Discovery 2018; 11:1390). Multiple lines of evidence (including data from real world) suggest that ESR1m is also associated with poor treatment outcomes in terms of both PFS and OS, mainly owing to lack of effective treatment options to address this driver mutation (Lei et al., J. Cancer Metastasis Treat. 2019; 5:38). Emergence of ESR1m is also associated with more aggressive disease features, including development of visceral metastases (see e.g. Reinert T. et al. Front Oncol 2017; 7:26).

The CDK4/6 cell cycle pathway, consisting of cyclin D-CDK4/6-INK4-Rb (Rb, the retinoblastoma protein), is frequently mutated in breast cancer, with pathway overactivation causing cells to erroneously progress through the G1/S checkpoint and proliferate. Estrogen itself is mitogenic, leads to increases in both cyclin D1 and CDK4/6 activity, and promotes excess proliferation in hormone-regulated breast cancers. Rb is a tumor suppressor protein that, when bound to the E2 transcription factor (E2F), prevents cell cycle progression. Targeting of CDK4/6 is thus indicated as a desirable option for intervening in the overactivated cyclin D-CDK4/6-INK4-Rb pathway in HR+ breast cancer as it allows Rb to remain functional and help control cell growth. Accordingly CDK4/6 inhibitors have been found to be particularly useful agents for the treatment of advanced HR+, HER2− breast cancer and have been widely adopted in clinical practice, most commonly in combination with an ET. Targeting components of the cyclin D-CDK4/6-INK4-Rb pathway also helps to circumvent resistance to anti-estrogens.

While CDK4/6 inhibitors have proven to be highly effective in the clinic for ER+ breast cancer patients, intrinsic or developed resistance to these drugs is common. About 20% of breast cancer patients treated with CDK4/6 inhibitors never respond to treatment. These patients' tumors already have mutations present that allow them to circumvent that action of the CDK4/6 inhibitor and proliferate in the presence of the drug. This intrinsic resistance to CDK4/6 inhibitors commonly involves the activation of the cyclin D-CDK4/6-Rb pathway. Acquired resistance to CDK4/6 inhibitors following initial response can manifest in a number of ways including cyclin D-CDK4/6-Rb activation, the activation of other proliferation pathways, the alteration of the tumor microenvironment, and the adjustment of the tumor metabolism. Within 2 years of beginning treatment in the PALOMA-2 trial, over 30% of enrolled patients developed resistance to the CDK4/6 inhibitor palbociclib. Treatment of CDK4/6 resistant HR+/HER2− breast cancer represents a significant unmet medical need.

Next-generation oral selective estrogen receptor degraders (ngSERDs) are aiming to become the backbone endocrine therapy for patients with HR+ breast cancer by delivering greater ER signalling blockade than current therapies and tackling key mechanisms of resistance. Camizestrant (AZD9833) is a ngSERD for the treatment of ER+ breast cancer which has demonstrated selective ERα degradation, pure ER antagonism and significant anti-tumour activity in ESR1 wild-type (ESR1wt) and mutant (ESR1m) tumors, as well as encouraging clinical activity in early phase clinical trials.

As noted above current guidelines recommend combining an endocrine therapy, such as an Al or a SERD, with an inhibitor of cyclin-dependent kinases 4 and 6 (CDK4/6i) as first line (1L) treatment for HR+/HER− breast cancer (see e.g. Cardoso F et al. Ann. Oncol. 2020; S0923-7534, (20), 42460-3). Unfortunately, most patients eventually experience cancer progression on CDK4/6 inhibitor+ET and succumb to their disease. Once a patient progresses on 1L CDK4/6i based therapy, subsequent endocrine-based therapy may have limited efficacy and challenging tolerability profiles. Many patients are eventfully treated with chemotherapy. However, drug resistance eventually develops, leading to disease progression (i.e. where the cancer's growth is not sufficiently, or not all, controlled by previously administered drug regimens, for example as assessed on the basis of radiological or other analytical means).

In addition to intrinsic and acquired resistance profiles, the presence of lung and/or liver metastases is an important prognostic factor in advanced breast cancer indicating aggressive and hard to treat disease.

Accordingly, there is a need for improved therapeutic approaches to the treatment of HR+, HER2− breast cancer, in particular in those patients with locally advanced or metastatic breast cancer that has recurred or progressed following at least one prior line of endocrine therapy. It is an object of the specification to provide a new method of treatment for such patients.

### SUMMARY

In a first aspect the present specification provides a method of treatment of hormone receptor positive (HR+), human epidermal growth factor receptor 2 negative (HER2−), metastatic or loco-regionally recurrent breast cancer, comprising administering a therapeutically effective amount of a next generation selective estrogen receptor degrader (ngSERD) to a patient suffering from such cancer, characterised in that the cancer has recurred or progressed following at least one prior line of endocrine therapy.

In this and other aspects detailed below the “recurrence” or “progression” of a cancer is following prior treatment with an endocrine therapy (ET), optionally a SERM or an aromatase inhibitor. The patient for treatment may also have received chemotherapy and/or a CDK4/6 inhibitor therapy and will have received no more than one line of ET or chemotherapy for the treatment of advanced disease. CDK4/6 inhibitors are usually administered in combination with ET, for example in combination with an Al.

The terms “treat,” “treating,” and “treatment” refer to at least partially alleviating, inhibiting, preventing and/or ameliorating a condition, disorder, or disease, such as breast cancer. The term “treatment of cancer” includes both in-vitro and in-vivo treatments, including in warm-blooded animals such as humans. The effectiveness of treatment of cancer can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival. Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena. Other assessments of effectiveness are known to those of skill in the art.

The term “therapeutically effective amount” refers to that amount of a compound or combination of compounds as described herein that is sufficient to effect the intended application including, but not limited to, disease treatment. A therapeutically effective amount may vary depending upon the intended application (in vitro or in-vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g. the amount of apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

“Hormone receptor-positive (HR+), human epidermal growth factor receptor 2 negative (HER2−) breast cancer” refers to tumours that express one or both of the estrogen receptor (ER) and/or the progesterone receptor (PgR) (i.e. that are HR+), and that have low levels of, or an absence of, the human epidermal growth factor receptor on their cell surface (i.e. that are HER2−). These terms are well known to those skilled in the art.

In related aspects the present specification provides a ngSERD for use in the treatment of HR+, HER2− metastatic or loco-regionally recurrent breast cancer, characterised in that the breast cancer has recurred or progressed following at least one prior line of endocrine therapy.

In related aspects the present specification provides a ngSERD for use in the manufacture of a medicament for the treatment of HR+, HER2−, metastatic or loco-regionally recurrent breast cancer wherein the medicament is for use in the treatment of a breast cancer that has recurred or progressed following at least one prior line of endocrine therapy.

In aspects according to the specification, the breast cancer has recurred or progressed following a least one line of endocrine therapy, optionally where in the endocrine therapy is selected from AI or SERM therapy.

In aspects according to the specification, the breast cancer has recurred or progressed following a least one line of therapy with a CDK4/6 inhibitor. In such aspects the CDK4/6 inhibitor may have been administered in combination with an endocrine therapy, for example an aromatase inhibitor.

In aspects according to the present specification, the patient has a breast cancer has a mutation on the estrogen receptor (ESR1m) , i.e. the breast cancer is a ESR1m breast cancer.

In aspects according to the present specification, the patient has visceral metastases (for example metastases to the liver and/or the lungs).

In aspects according to the present specification, the ngSERD for use in treatment, in a method of treatment, or in a method of manufacture of a medicament for use in treatment is camizestrant (AZD9833, N-(1-(3-fluoropropyl)azetidin-3-yl)-6-((6S,8R)-8-methyl-7-(2,2,2-trifluoroethyl)-6,7,8,9-tetrahydro-3H-pyrazolo[4,3-f]isoquinolin-6-yl)pyridin-3-amine) or a pharmaceutically acceptable salt thereof. In such aspects the camizestrant or a pharmaceutically acceptable salt thereof may be administered orally at a dose of 75 mg or 150 mg per day. In some aspects the ngSERD for use in treatment, in a method of treatment, or in a method of manufacture of a medicament for use in treatment is camizestrant in its non-salt form (i.e. as its free base).

In aspects according to the present specification, the ngSERD for use in treatment, for use in a method of treatment or for use in the manufacture of a medicament provides an improvement in progression free survival (PFS) relative to that observed with standard of care (SoC). In such aspects the SoC may be fulvestrant.

In aspects according to the present specification, the ngSERD for use in treatment, for use in a method of treatment or in medicine comprising a ngSERD provides an improvement in overall survival (OS) relative to standard of care.

In aspects according to the present specification, the ngSERD for use in treatment, for use in a method of treatment or in medicine comprising a ngSERD provides an improvement in objective response rate (ORR) relative to standard of care.

In aspects according to the present specification, the ngSERD for use in treatment, for use in a method of treatment or in medicine comprising a ngSERD provides an improvement in clinical benefit rate at 24 weeks (CBR₂₄) relative to standard of care.

In aspects according to the present specification, the ngSERD for use in treatment, for use in a method of treatment or in medicine comprising a ngSERD causes a clearance of mutation in the estrogen receptor of the tumor. In such aspects the clearance of ESR1m is measured in a blood sample analysed for circulating tumor DNA or is measured in a tumor biopsy obtained from the patient. In such aspects the presence of ESR1m may be identified by analysis of a tumor biopsy. In such aspects the clearance of ESR1m is measured in a blood sample obtained from the patient and analysed for circulating tumor DNA. In such aspects the presence of ESR1m may be identified by analysis of a tumor biopsy. In such aspect the clearance of mutation in the estrogen receptor is increased relative to that observed with treatment with fulvestrant.

In aspects according to the present specification, the ngSERD for use in treatment, for use in a method of treatment or in medicine comprising a ngSERD does not cause any clinically significant treatment related adverse effects (TRAEs).

In an aspect according to the present specification, there is provided a kit comprising a medicament comprising a ngSERD and instructions for use of the medicament in the treatment of HR+/HER2− metastatic or loco-regionally recurrent breast cancer in a patient whose cancer has recurred or progressed following at least one prior line of endocrine therapy.

In an aspect according to the present specification, there is provided a pharmaceutical composition comprising a ngSERD and at least one pharmaceutically acceptable excipient for use in the treatment of HR+/HER2− metastatic or loco-regionally recurrent breast cancer in a patient whose cancer has recurred or progressed following at least one prior line of endocrine therapy.

### FIGURES

So that the invention may be more readily understood reference is made herein to the following Figures.

FIG. 1: Kaplan Meyer curve showing the probability of progression free survival as assessed by Investigator vs time on treatment (in months) with fulvestrant ((F) 500 mg, once monthly injection) or camizestrant ((C) administered as a 75 mg or 150 mg tablet once per day). Both doses of camizestrant are seen to deliver a clinically meaningful improvement in PFS over fulvestrant. The proportion of patients without progression at 12 m was 23.8%, 34.3%, and 44.5% for fulvestrant, 75 mg camizestrant, and 150 mg camizestrant, respectively.

FIG. 2: Kaplan Meyer curve showing the probability of progression free survival as assessed by Blinded Independent Central Review (BICR) vs time on treatment (in months) with fulvestrant ((F) 500 mg, once monthly injection) or camizestrant ((C) administered as a 75 mg or 150 mg tablet once per day). Both doses of camizestrant are seen to deliver a clinically meaningful improvement in PFS over fulvestrant. Discordance at the patient level for progression between BICR and Investigator assessment are consistent with those observed in general (see K Borradaile et al., Cancer Research 2009: 62 (2 Suppl): Abstract No. 2081).

FIG. 3: Kaplan Meyer curve showing the probability of progression free survival vs time (in months) for patients that had received prior CDK4/6 inhibitor treatment. In this subgroup that received prior treatment with CDK4/6i, both 75 mg and 150 mg camizestrant treatment produced a comparable improvement over fulvestrant treatment with HRs of 0.49 and 0.68, respectively.

FIG. 4: Kaplan Meyer curve showing the probability of progression free survival vs time (in months) for patients that had lung and/or liver metastases before treatment commenced. In this subgroup both 75 mg and 150 mg camizestrant treatment produced a comparable improvement over fulvestrant treatment with HRs of 0.43 and 0.55, respectively.

FIG. 5: Kaplan Meyer curve showing the probability of progression free survival vs time (in months) for patients that had mutations to the estrogen receptor (ESR1m) before treatment commenced. In this subgroup both 75 mg and 150 mg camizestrant treatment produced a comparable improvement over fulvestrant treatment with HRs of 0.33 and 0.55, respectively.

FIG. 6: Changes in ESR1m ctDNA summed Variant Allele Frequency (sVAF %) by treatment group and visit comparisons (pre-treatment is a comparison of samples collected at screening and cycle 1 day 1, C×D1 are comparisons of samples collected at cycle 1 day 1 and cycle×day 1). Variants defined as ESR1m are E380Q, V422del, S463P, L536H/P/R, Y537C/D/N/S. Points represent individual patients, box represents upper quartile, median and lower quartile, whiskers represent 1.5 interquartile range. Treatment with camizestrant 75 mg and 150 mg caused a 100% or near 100% reduction in the level of ESR1m at all timepoints. In contrast, although reduction in ESR1m ctDNA is observed in the fulvestrant arm, the extent of the reduction is lower than that observed with camizestrant.

### DETAILED DESCRIPTION

Estrogen receptor-α is a well-established drug target in breast cancer with ETs being the mainstay of treatment. ETs include SERMs (e.g. tamoxifen), SERDs (fulvestrant), and AIs (e.g. the non-steroidal AIs anastrozole and letrozole, and the steroidal AI exemestane). It is well established that CDK4/6 inhibitors (palbociclib, ribociclib or abemaciclib) enhance ET efficacy in untreated and previously treated HR+/HER2− metastatic breast cancer (MBC) by extending progression free survival (PFS) and overall survival (OS). Current treatment guidelines recommend Al in combination with CDK4/6 inhibitor as the standard of care in the first line (1L) HR+/HER2− MBC setting for most postmenopausal women, premenopausal women in combination with a LHRH agonist, and men, preferably in combination with an LHRH agonist, across many regions. At the date of writing three CDK4/6 inhibitors are approved for the treatment of HR+, HER2− breast cancer, namely palbociclib, abemaciclib and ribociclib.

Unfortunately, most patients eventually have cancer progression on CDK4/6 inhibitor+AI and succumb to their disease. Once patients progress on 1L CDK4/6 inhibitor based therapy, the subsequent endocrine-based therapies have limited efficacy with challenging tolerability profile. Most patients may eventfully be treated with chemotherapy. There is therefore the need for improved therapies for patients who progress following treatment with an AI and a CDK4/6i. Treatment of HR+, HER2− advanced breast cancer patients that are CDK4/6 inhibitor resistant, that have tumors that have a mutation to the estrogen receptor (i.e. that have ESR1m status) or that have tumors that have metastasized to the lung and/or liver (or that have these and additional visceral metastases) is particularly challenging. It is an object of the present specification to provide new treatment option for such patient groups.

In a first embodiment the present specification provides a method of treatment of hormone receptor positive (HR+), human epidermal growth factor receptor 2 negative (HER2−), metastatic or loco-regionally recurrent breast cancer, comprising administering a therapeutically effective amount of a next generation selective estrogen receptor degrader (ngSERD) to a patient suffering from such cancer, characterised in that the cancer has recurred or progressed following at least one prior line of endocrine therapy. In embodiments the prior endocrine therapy is selected from aromatase or SERM therapy, optionally wherein the prior treatment has comprised administration of an aromatase inhibitor selected from anastrazole, letrazole or exemestane and/or a selective estrogen receptor modulator (SERM) such as tamoxifen.

In a related embodiment the present specification provides a ngSERD for use in the treatment of HR+, HER2− metastatic or loco-regionally recurrent breast cancer, characterised in that the breast cancer has recurred or progressed following at least one prior line of endocrine therapy.

In a related embodiment the present specification provides a ngSERD for use in the manufacture of a medicament for the treatment of HR+, HER2−, metastatic or loco-regionally recurrent breast cancer wherein the medicament is for use in the treatment of a breast cancer that has recurred or progressed following at least one prior line of endocrine therapy. In a related embodiment the present specification provides a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy.

In a related embodiment the present specification provides a pharmaceutical composition comprising a ngSERD and at least one pharmaceutically acceptable excipient for use in the treatment of HR+/HER2− metastatic or loco-regionally recurrent breast cancer in a patient whose cancer has recurred or progressed following at least one prior line of endocrine therapy.

For the avoidance of doubt, embodiments provided below relate to further characterising features of embodiments relating to the method of treatment, ngSERD for use, ngSERD for use in the manufacture of a medicament for treatment, kit and composition for use in embodiments elsewhere in the specification (for example those directly above) and their features may be incorporated into these general embodiments. For example, the reader will understand that embodiments relating to a method of treatment with certain characterising features can read on to a ngSERD for use in treatment of the same condition in patients with the same characterising features and outcomes et cetera.

As noted above SERDs bind to the estrogen receptor causing it to be degraded and therefore downregulated. The first generation SERD, fulvestrant, is administered as a once monthly injection and it is hypothesized that next generation oral SERDs may provide benefit for the treatment of HR+, HER2− breast cancer by providing a greater net degradation of the estrogen receptor resulting from more frequent administration and higher net systemic concentrations.

In embodiments the ngSERD may be selected from camizestrant or a pharmaceutically acceptable salt thereof, giredestrant or a pharmaceutically acceptable salt thereof, imlunestrant or a pharmaceutically acceptable salt thereof and elacestrant or a pharmaceutically acceptable salt thereof. In embodiments of the present specification the ngSERD may be used in free base form or in the form of a pharmaceutically acceptable salt or prodrug. The term “pharmaceutically acceptable” is used herein to specify that an object (for example a salt, dosage form [such as a tablet or capsule] or excipient [such as a diluent or carrier]) is suitable for use in patients. An example list of pharmaceutically acceptable salts can be found in the “Handbook of Pharmaceutical Salts: Properties, Selection and Use”, P. H. Stahl and C. G. Wermuth, editors, Weinheim/Zurich:Wiley-VCH/VFICA, 2002 or subsequent editions.

In embodiments the ngSERD is camizestrant or a pharmaceutically acceptable salt thereof.

In embodiments the ngSERD is giredestrant or a pharmaceutically acceptable salt thereof.

In embodiments the ngSERD is imlunestrant or a pharmaceutically acceptable salt thereof.

In embodiments, an ER PROTAC may be used in place of a ngSERD. In embodiments the ER PROTAC may be ARV-471.

Camizestrant (AZD9833) has the following chemical structure:

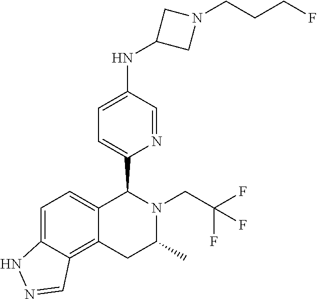 [SMILES: C[C@@H]1Cc2c(ccc3[nH]ncc23)[C@@H](c2ccc(NC3CN(CCCF)C3)cn2)N1CC(F)(F)F]

The free base of camizestrant is known by the chemical name N-(1-(3-fluoropropyl)azetidin-3-yl)-6-((6S,8R)-8-methyl-7-(2,2,2-trifluoroethyl)-6,7,8,9-tetrahydro-3H-pyrazolo[4,3-f]isoquinolin-6-yl)pyridin-3-amine. Camizestrant is disclosed in WO2018077630A1.

Giredestrant (GDC-9545) has the following chemical structure:

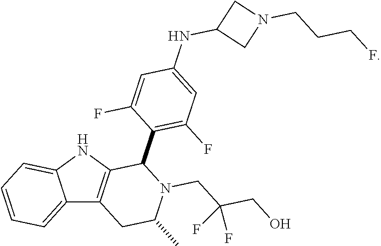 [SMILES: C[C@@H]1Cc2c([nH]c3ccccc23)[C@@H](c2c(F)cc(NC3CN(CCCF)C3)cc2F)N1CC(F)(F)CO]

The free base of giredestrant is known by the chemical name 3-[(1R,3R)-1-[2,6-difluoro-4-[[1-(3-fluoropropyl)azetidin-3-yl]amino]phenyl]-3-methyl-1,3,4,9-tetrahydropyrido[3,4-b]indol-2-yl]-2,2-difluoropropan-1-ol. Giredestrant is disclosed in WO2016097072A1.

Imlunestrant (LY-3484356) has the following chemical structure:

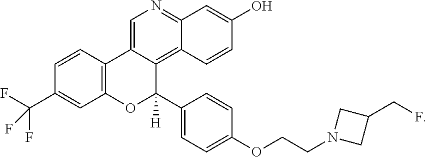 [SMILES: Oc1ccc2c3c(cnc2c1)-c1ccc(C(F)(F)F)cc1O[C@@H]3c1ccc(OCCN2CC(CF)C2)cc1]

The free base of imlunestrant is known by the chemical name (5R)-5-[4-[2-[3-(fluoromethyl)azetidin-1-yl]ethoxy]phenyl]-8-(trifluoromethyl)-5H-chromeno[4,3-c]quinolin-2-ol. Imlunestrant is disclosed in WO2020014435.

ARV-471, disclosed in WO2018102725, has the following chemical structure:

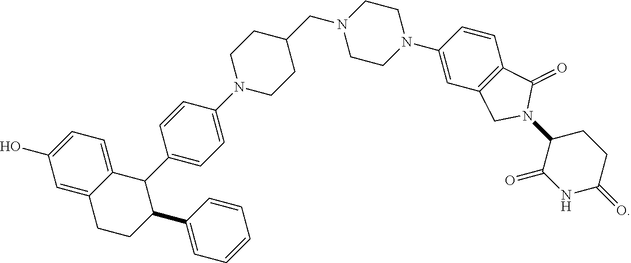

In embodiments camizestrant or a pharmaceutically acceptable salt thereof is administered to the subject at a daily dosage of 75 mg or 150 mg.

The nature of the HR+, HER2−, metastatic or loco-regionally recurrent breast cancer may be further defined in terms of the prior treatment that the patient has received, the mutation status of the cancer, the response profile of that the cancer exhibited to prior endocrine therapy or the presence of visceral metastases (such as the presence of metastases in the liver and/or lung). Such parameters can be prognostic of the response to therapy.

In embodiments of the present speciation the method of treatments, ngSERD for use in treatment or for use in the manufacture of a medicament, kit or pharmaceutical composition for use delivers an improvement in the time to disease progression or progression free survival (PFS) relative to that obtained from use of fulvestrant (the first generation SERD). In such embodiment the PFS improvement is relative to that observed with standard of care, i.e. fulvestrant administered at the approved dose of 500 mg once monthly by injection. Clinical activity as assessed by PFS can be determined by the standard assessment of tumour response as per RECIST 1.1 (Eisenhauser et al., Eur J Cancer 2009 January; 45(2):228-47). The RECIST criteria can be evaluated by the treating physician/investigator or by Blinded Independent Central Review (BICR).

In the SERENA-2 clinical trial, further details of which are provided below, the efficacy of camizestrant administered once daily in tablet form at doses of 75 mg and 150 mg as a treatment for patients with HR+, HER2− metastatic or loco-regionally recurrent breast cancer that the breast cancer has recurred or progressed following at least one prior line of endocrine therapy was assessed. The control arm patient in this study were treated with standard of care fulvestrant, the first generation SERD, administered as once monthly 500 mg injection. The study was powered to differentiate between the camizestrant and standard of care, fulvestrant (F), treatment arms, but was not powered to differentiate between the 75 mg and 150 mg camizestrant dose cohorts.

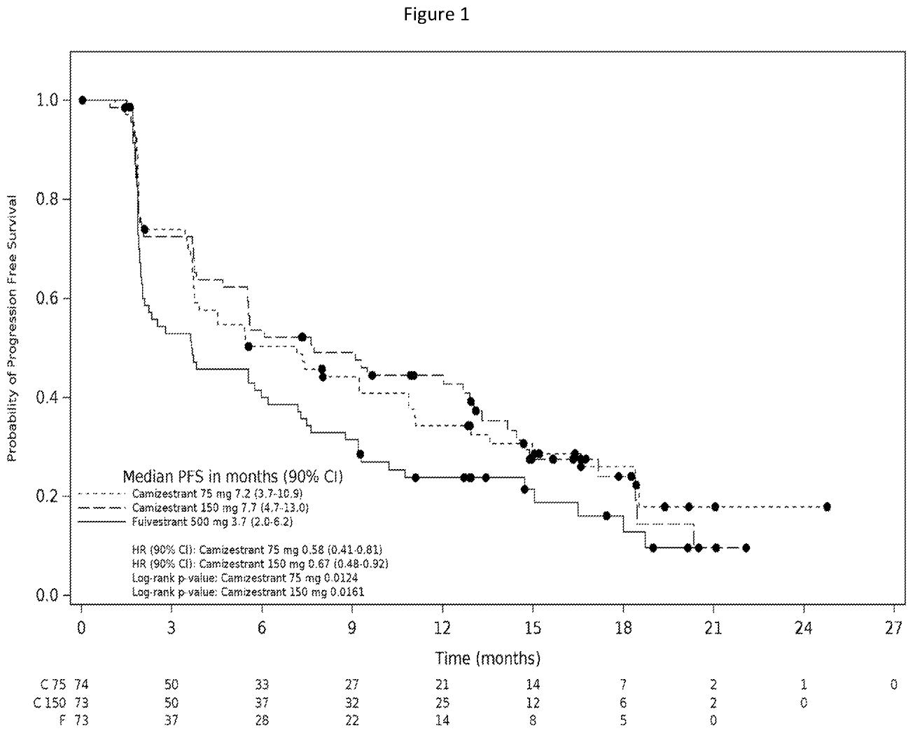

Fulvestrant as used in this study as standard of care comparator arm delivered a median PFS of 3.7 months (90% CI). In contrast the 75 mg and 150 mg camizestrant arms delivered median PFS values of 7.2 and 7.7 months (90% CI) and hazard ratios of 0.58 (p=0.0124) and 0.67 (p=0.0161), respectively as determined by Investigator (see FIG. 1). The proportion of patients without progression at 12 m was 23.8%, 34.3%, 44.5% for fulvestrant, 75 mg camizestrant, and 150 mg camizestrant respectively. It was thus established that a clinically meaningful improvement in PFS was achieved for both the camizestrant treatment arms relative to fulvestrant across all patients in the clinical trial irrespective of pre-treatment, estrogen receptor mutation status (ESR1m) or the presence of visceral metastases. In embodiments the PFS by the RECIST 1.1 criteria is determined by the treating physician or investigator.

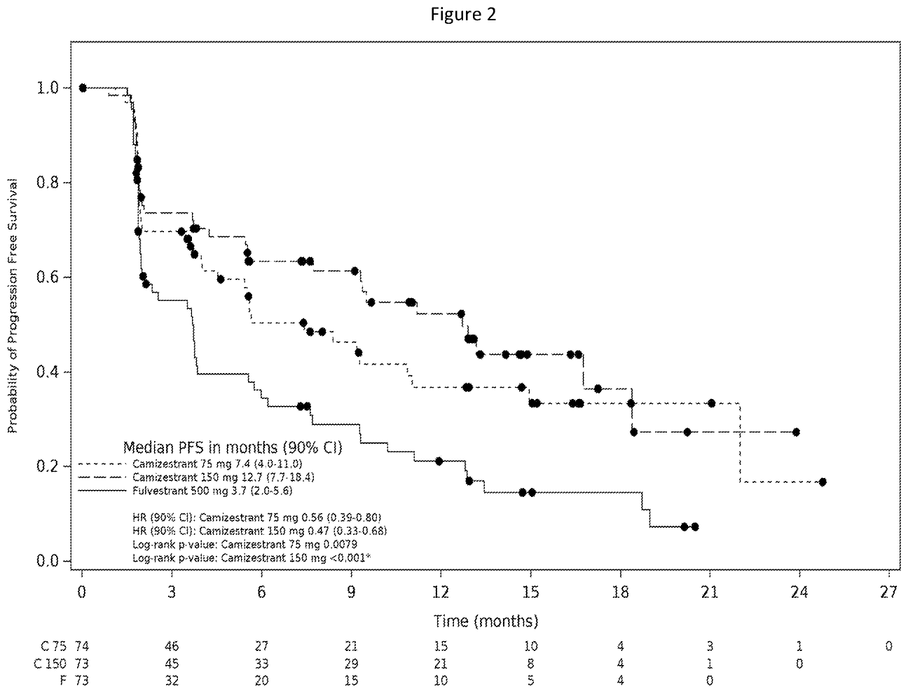

When PFS in the SERENA-2 study was evaluated according to the RECIST 1.1 criteria by Blinded Independent Central Review Fulvestrant, as used in this study as standard of care comparator arm, delivered a median PFS of 3.7 months (90% CI). In contrast the 75 mg and 150 mg camizestrant arms delivered median PFS values of 7.4 and 12.7 months (90% CI) and hazard ratios of 0.56 (p=0.0079) and 0.47 (p<0.001), respectively as determined by BICR (see FIG. 2).

The improvement in PFS observed under the RECIST 1.1 criteria by either Investigator or BICR therefore indicate an improvement of PFS in the all comer population in the SERENA-2 study of at least 3.5 months in both camizestrant arms (75 mg and 150 mg). As noted above, the study was not powered to differentiate between camizestrant arms, but was powered to differentiate between camizestrant treatment arms and the fulvestrant arms.

Accordingly in embodiments the present specification provides method of treatment of hormone receptor positive (HR+), human epidermal growth factor receptor 2 negative (HER2−), metastatic or loco-regionally recurrent breast cancer, comprising administering a therapeutically effective amount of a next generation selective estrogen receptor degrader (ngSERD) to a patient suffering from such cancer, characterised in that the cancer has recurred or progressed following at least one prior line of endocrine therapy, wherein the method extends the time to disease progression relative to that observed with fulvestrant treatment.

Accordingly in embodiments the present specification provides method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, wherein the method extends progression free survival relative to that observed with fulvestrant treatment.

in embodiments the present specification provides method of treatment of hormone receptor positive (HR+), human epidermal growth factor receptor 2 negative (HER2−), metastatic or loco-regionally recurrent breast cancer, comprising administering a therapeutically effective amount of camizestrant to a patient suffering from such cancer, characterised in that the cancer has recurred or progressed following at least one prior line of endocrine therapy, wherein the method extends progression free survival relative to that observed with fulvestrant treatment and wherein the hazard ratio for disease progression relative to fulvestrant is 0.67 or less. In such embodiments the hazard ratio for disease progression for camizestrant treatment relative to fulvestrant is 0.58 or less, for example 0.56 or less or 0.47 less. The hazard ratios obtained for camizestrant treatment relative to fulvestrant in the SERENA-2 trial as detailed herein are all statistically significant and are associated with a clinically meaningful improvement in progression free survival relative to that observed with treatment with standard of care.

In embodiments the present specification provides method of treatment of hormone receptor positive (HR+), human epidermal growth factor receptor 2 negative (HER2−), metastatic or loco-regionally recurrent breast cancer, comprising administering a therapeutically effective amount of camizestrant to a patient suffering from such cancer, characterised in that the cancer has recurred or progressed following at least one prior line of endocrine therapy, wherein the time to disease progression is at least 5 months, for example 6 months or more, 7 months or more, 8 months or more, 9 months or more or 12 months or more. The time to disease progression obtained from camizestrant treatment is statistically significant and are meaningful improvements on that observed with treatment with standard of care, fulvestrant its authorized dose.

Accordingly, in embodiments the specification provides methods of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, wherein the camizestrant is administered once daily at a dose of 75 mg or 150 mg and wherein the hazard ratio for disease progression derived from camizestrant treatment relative to fulvestrant treatment is 0.67 or less, 0.58 or less, 0.56 or less or 0.47 or less.

In embodiments the specification provides methods of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, and wherein the median progression free survival delivered by camizestrant treatment is at least 5 months, for example 6 months or more, 7 months or more, 8 months or more, 9 months or more, or 12 months or more.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, wherein the method delivers an improvement in progression free survival. In embodiments the improvement in progression free survival is relative to that obtainable with fulvestrant therapy. In embodiments the extension in PFS derived from use of camizestrant relative to use of fulvestrant is at least 1 month, for example 2 months or more, 3 months or more, or 4 months or more.

In embodiments the specification provides methods of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, and wherein the median time to disease progression obtained from treatment with camizestrant relative to that observed with fulvestrant treatment is extended by a period of at least two months, optionally at least 3.5 months.

In embodiments where the method of treatment provides an improvement in PFS, the progression of disease is evaluated on the basis of the RECIST 1.1 criteria. In embodiments the evaluation of disease progression by the RECIST 1.1 criteria is performed by the Investigator or treating physician. In embodiments the evaluation of disease progression by the RECIST 1.1 criteria is performed by Blinded Independent Central Review (BICR).

In embodiments the at least one prior line of endocrine therapy may be aromatase inhibitor therapy, i.e. therapy with anastrazole or letrazole or exemestane. In embodiments the at least one prior line of endocrine therapy may be SERM therapy, optionally tamoxifen therapy.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, and wherein the treatment delivers an improvement in overall survival.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, wherein the treatment delivers an improvement in objective response rate (ORR) relative to that obtainable with fulvestrant treatment. The ORR is defined as the percentage of patients with at least 1 Investigator-assessed visit response of complete response (CR) or partial response (PR) prior to any evidence of progression. Complete or partial responses are assessed according to the RECIST 1.1 criteria as detailed below. In embodiments the ORR derived from the method of treatment is at least 15.7%, for example 20.3%. ORR data obtained from the SERENA-2 study is provided in Table 1 below.

A complete response denotes the disappearance of all target lesions (TLs) since baseline. Any pathological lymph nodes selected as TLs must have a reduction in short axis to <10 mm. A partial response denotes an at least a 30% decrease in the sum of the diameters of TL, taking as reference the baseline sum of diameters. Other evaluations of target lesions include stable disease (SD) that denotes neither sufficient shrinkage to qualify for PR nor sufficient increase to qualify for progressive disease (PD) and progressive disease that indicates at least a 20% increase in the sum of diameters of TLs, taking as reference the smallest sum on study (this includes the baseline sum if that is the smallest on study) and, in addition to the relative increase of 20%, the sum must also demonstrate an absolute increase of at least 5 mm. Further information on the evaluation of target and non-target lesions is provided below.

TABLE 1
Objective Response Rate, patients with measurable disease.

| | | | | | | Comparison against | | |
|---|---|---|---|---|---|---|---|---|
| | | | Number (%) | | Adjusted | fulvestrant | | |
| | | | of patients | | response | Odds | 90% | 2-sided |
| Group | N | NE | with response | | rate (%) | ratio | CI | p-value |
| AZD9833 | 70 | 4 | 11 | (15.7) | 15.7 | 1.43 | 0.63- | 0.4789 |
| 75 mg | | | | | | | 3.33 | |
| AZD9833 | 65 | 5 | 13 | (20.0) | 20.3 | 1.96 | 0.88- | 0.1675 |
| 150 mg | | | | | | | 4.51 | |
| Fulves- | 68 | 3 | 8 | (11.8) | 11.5 | | | |
| trant | | | | | | | | |

The analysis was performed using logistic regression with factors for prior use of CDK 4/6 inhibitors and presence of lung and/or liver metastasis.
An odds ratio > 1 favors AZD9833 (camizestrant).
Responses include unconfirmed responses. RECIST version 1.1.
NE = Not Evaluable. NE represents patients with measurable disease at baseline without post-baseline RECIST scans. N = number of patients in group.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, and wherein the treatment delivers an improvement in clinical benefit rate at 24 weeks (CBR₂₄) relative to that obtained from fulvestrant treatment.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy, and wherein the treatment delivers a clinical benefit rate at 24 weeks (CBR₂₄) of at least 48%, for example 52%.

The CBR₂₄ is defined as the percentage of patients who have a best objective response (BoR) of CR or PR in the first 25 weeks (to allow for a late assessment within the assessment window) or who have SD (without subsequent cancer therapy) for at least 23 weeks after start of treatment (to allow for an early assessment within the assessment window). The CBR is defined based on the Investigator's assessment of RECIST 1.1.

TABLE 2
Clinical Benefit Rate at 24 weeks (CBR₂₄)

| | | Number (%) of | Adjusted | Comparison against | | |
|---|---|---|---|---|---|---|
| | | patients with | CBR24 | fulvestrant | | |
| | | clinical benefit | rate | Odds | 90% | 2-sided |
| Group | N | at 24 weeks | (%) | ratio | CI | p-value |
| AZD9833 | 70 | 34 (48.6) | 50.4 | 1.73 | 0.96-3.15 | 0.1294 |
| 75 mg | | | | | | |
| AZD9833 | 65 | 34 (52.3) | 55.2 | 2.09 | 1.14-3.87 | 0.0437 |
| 150 mg | | | | | | |
| Fulves- | 68 | 25 (36.8) | 37.1 | | | |
| trant | | | | | | |

The analysis was performed using logistic regression with factors for prior use of CDK4/6 inhibitors and presence of lung and/or liver metastasis.
An odds ratio > 1 favors AZD9833 (camizestrant). Responses included unconfirmed responses. RECIST version 1.1.
Patients evaluable for clinical benefit defined as patients randomized for at least 25 weeks prior to analysis, or who had their 24 week RECIST assessment. N = number of patient evaluable.
Clinical benefit defined as patients with best objective response of complete response or patient response in the first 25 weeks or who have stable disease for at least 23 weeks after randomization.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and one prior line of therapy with a CDK4/6 inhibitor. In embodiments the patient's disease has recurred or progressed following therapy with palbociclib. In embodiments the patient's disease has recurred or progressed following therapy with abemaciclib. In embodiments the patient's disease has recurred or progressed following therapy with ribociclib.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and one prior line of therapy with a CDK4/6 inhibitor, wherein the median time to progression is extended by at least three months relative to that observed with fulvestrant treatment.

In embodiments the specification provides a method of treatment of hormone receptor positive (HR+), human epidermal growth factor receptor 2 negative (HER2−), metastatic or loco-regionally recurrent breast cancer comprising administering camizestrant to a patient in need thereof, characterised in that the patient's cancer has recurred or progressed following at least one prior line of endocrine therapy and one prior line of therapy with a CDK4/6 inhibitor, wherein the hazard ratio for disease progression derived from camizestrant treatment relative to fulvestrant treatment is 0.68 or less, for example 0.49.

In embodiments the specification provides a method of treatment of hormone receptor positive (HR+), human epidermal growth factor receptor 2 negative (HER2−), metastatic or loco-regionally recurrent breast cancer comprising administering camizestrant to a patient in need thereof, characterised in that the patient's cancer has recurred or progressed following at least one prior line of endocrine therapy and one prior line of therapy with a CDK4/6 inhibitor, wherein the time to disease progression is at least 3.8 months , for example 5.8 months.

TABLE 3
Progression Free Survival for patients with disease that has progressed
or recurred following at least one prior line of endocrine therapy
and one prior line of therapy with a CDK4/6 inhibitor

| | Camizestrant | Camizestrant | Fulvestrant |
|---|---|---|---|
| | 75 mg | 150 mg | 500 mg |
| | (n = 38) | (n = 37) | (n = 37) |
| Events | 29 | 29 | 33 |
| [n (%)] | (76.3) | (78.4) | (89.2) |
| Median PFS, | 5.5 | 3.8 | 2.1 |
| months | (3.7-10.9) | (2.0-7.6) | (1.9-3.7) |
| (90% CI) | | | |
| Hazard ratio | 0.49 | 0.68 | — |
| (90% CI) | (0.31-0.75) | (0.44-1.04) | |
| 2-sided | 0.0064* | 0.0628* | — |
| P value | | | |

n = number of patients in each arm with stated characteristic

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and one prior line of therapy with chemotherapy.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has received no prior treatment with fulvestrant, any other oral SERD, or any related therapies (e.g. ER proteolysis targeting chimeras [PROTACs] and/or selective ER covalent antagonists [SERCAs] in the metastatic setting).

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has visceral metastases prior to commencing camizestrant treatment.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having metastases to the liver and/or lungs prior to commencing camizestrant treatment.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having metastases to the liver and/or lungs prior to commencing camizestrant treatment, wherein the median time to progression is extended by at least three months relative to that observed with fulvestrant treatment, for example by 3.6 months or 5.2 months.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having metastases to the liver and/or lungs prior to commencing camizestrant treatment, wherein the hazard ratio for disease progression derived from camizestrant treatment relative to fulvestrant treatment is 0.55 or less, for example 0.43.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having metastases to the liver and/or lungs prior to commencing camizestrant treatment, wherein the time to disease progression is at least 5.6 months , for example 7.2 months.

TABLE 4
Progression Free Survival for patients with disease that has
progressed or recurred following at least one prior line of
endocrine therapy and that have metastases to the liver and/or
lungs prior to commencement of treatment with camizestrant

| | Camizestrant | Camizestrant | Fulvestrant |
|---|---|---|---|
| | 75 mg | 150 mg | 500 mg |
| | (n = 43) | (n = 43) | (n = 43) |
| Events | 31 | 32 | 39 |
| [no. (%)] | (72.1) | (74.4) | (90.7) |
| Median PFS, | 7.2 | 5.6 | 2.0 |
| months | (3.6-11.1) | (3.7-9.1) | (1.9-3.6) |
| (90% CI) | | | |
| Hazard ratio | 0.43 | 0.55 | — |
| (90% CI)* | (0.28-0.65) | (0.37-0.82) | |
| 2-sided | 0.0011 | 0.0061 | |
| P value | | | |

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having a breast cancer with a mutation to the estrogen receptor. In embodiments a mutation to the estrogen receptor is selected from E380Q, V422del, S463P, L536H, L536P, L536R, Y537C, Y537D, Y537N, Y537S and D538G.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the cancer has been identified as expressing ESR1m.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having a ESR1m breast cancer, wherein the median time to disease progression is extended by at least three months relative to that observed with fulvestrant treatment, for example by 4.1 months or 7.0 months.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having a ESR1m breast cancer, wherein the hazard ratio for disease progression derived from camizestrant treatment relative to fulvestrant treatment is 0.55 or less, for example 0.33.

In embodiments the specification provides a method of treatment of hormone receptor positive (HR+), human epidermal growth factor receptor 2 negative (HER2−), metastatic or loco-regionally recurrent breast cancer, comprising administering camizestrant to a patient suffering from such cancer, characterised in that the cancer has recurred or progressed following at least one prior line of endocrine therapy and the patient has been identified as having a ESR1m breast cancer, wherein the time to disease progression is at least 6.3 months , for example 9.2 months.

In embodiments the presence of a mutation to the estrogen receptor in the patient's cancer or identification of the ESR1m status of the breast cancer is made on the basis of a circulating tumor DNA test. In embodiments the presence of a mutation to the estrogen receptor in the patient's cancer or identification of the ESR1m status of the breast cancer is made on the basis of analysis of a tumor biopsy for the presence of ESR1m.

TABLE 5
Progression Free Survival for patients with disease
that has progressed or recurred following at least
one prior line of endocrine therapy and having a breast
cancer with a mutation to the estrogen receptor.

| | AZD9833 | AZD9833 | Fulvestrant |
|---|---|---|---|
| | 75 mg | 150 mg | 500 mg |
| | (n = 22) | (n = 26) | (n = 35) |
| Events | 15 | 22 | 31 |
| [no. (%)] | (68.2) | (84.6) | (88.6) |
| Median PFS, | 6.3 | 9.2 | 2.2 |
| months | (3.4-12.9) | (3.7-12.9) | (1.9-3.8) |
| (90% CI) | | | |
| Hazard ratio | 0.33 | 0.55 | — |
| (90% CI) | (0.18-0.58) | (0.33-0.89) | |
| 2-sided | 0.0047* | 0.0272* | — |
| P value | | | |

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy.

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy, and:
a) the use delivers an improvement to the median time to disease progression of at least 3.5 months relative to that observed with fulvestrant treatment; and/or
b) the hazard ratio for ngSERD treatment relative to fulvestrant treatment is less than or equal to 0.67; and/or
c) the use delivers a median time to disease progression of at least 7 months.
   In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy and at least one prior line of therapy with a CDK4/6 inhibitor.

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy and at least one prior line of therapy with a CDK4/6 inhibitor, and:
a) the use delivers an improvement to the median time to disease progression of at least 1.7 months more than that observed with fulvestrant treatment; and/or
b) the hazard ratio for ngSERD treatment relative to fulvestrant treatment is less than or equal to 0.68; and/or
c) the use delivers a median time to disease progression of at least 3.8 months.

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy and the cancer has been identified as having visceral metastases.

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy and the cancer has been identified as having visceral metastases, and:
a) the use delivers an improvement to the median time to disease progression of at least 3.6 months more than that observed with fulvestrant treatment; and/or
b) the hazard ratio for ngSERD treatment relative to fulvestrant treatment is less than or equal to 0.55; and/or
c) the use delivers a median time to disease progression of at least 5.6 months.

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy, and wherein the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G).

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy, and wherein the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G) on the basis of a test of a sample obtained from the patient (for example a test performed by analysing circulating tumor DNA), for example wherein the sample is a tumor biopsy or a blood sample.

In one embodiment the present specification provides a ngSERD (for example camizestrant or a pharmaceutically acceptable salt thereof which is optionally orally administered once daily at a dose of 75 mg or 150 mg) for use in the treatment of HR+, HER2− metastatic or loco-regionally recurrent breast cancer, characterised in that the breast cancer has recurred or progressed following at least one prior line of endocrine therapy and the use improves overall survival relative to treatment with fulvestrant.

In one embodiment the present specification provides camizestrant or a pharmaceutically acceptable salt thereof for use in the treatment of HR+, HER2−, metastatic or loco-regionally recurrent breast cancer, characterised in that the cancer has recurred or progressed following at least one prior line of endocrine therapy, wherein:
a) the use delivers an improvement to the median time to disease progression of at least 3.5 months relative to that observed with fulvestrant treatment; and/or
b) the hazard ratio for use of camizestrant treatment relative to fulvestrant treatment is less than or equal to 0.67; and/or
c) the use delivers a median time to disease progression of at least 7 months.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2−, metastatic or loco-regionally recurrent breast cancer, wherein the medicament is for use in the treatment of a breast cancer that has recurred or progressed following at least one prior line of endocrine therapy.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy, and wherein the medicament for use delivers:
a) an improvement in the median time to disease progression of at least 3.5 months relative to that observed with fulvestrant treatment; and/or
b) a hazard ratio for ngSERD treatment relative to fulvestrant treatment which is less than or equal to 0.67; and/or
c) a median time to disease progression of at least 7 months.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy and at least one prior line of therapy with a CDK4/6 inhibitor.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy and at least one prior line of therapy with a CDK4/6 inhibitor, and wherein the medicament for use delivers:
a) an improvement in the median time to disease progression of at least 1.7 months more than that observed with fulvestrant treatment; and/or
b) a hazard ratio for ngSERD treatment relative to fulvestrant treatment which is less than or equal to 0.68; and/or
c) a median time to disease progression of at least 3.8 months.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy and the cancer has been identified as having visceral metastases.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy and the cancer has been identified as having visceral metastases, and wherein the medicament for use delivers:
a) an improvement in the median time to disease progression of at least 3.6 months more than that observed with fulvestrant treatment; and/or
b) a hazard ratio for ngSERD treatment relative to fulvestrant treatment which is less than or equal to 0.55; and/or
c) a median time to disease progression of at least 5.6 months.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy, and wherein the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G).

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy, and wherein the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G) on the basis of a test of a sample obtained from the patient (for example a test performed by analysing circulating tumor DNA), for example wherein the sample is a tumor biopsy or a blood sample.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the breast cancer has recurred or progressed following at least one prior line of endocrine therapy and the use improves overall survival relative to treatment with fulvestrant.

In one embodiment the present specification provides a ngSERD (for example camizestrant or a pharmaceutically acceptable salt thereof which is optionally orally administered once daily at a dose of 75 mg or 150 mg) for use in the manufacture of a medicament for the treatment of HR+, HER2− metastatic or loco-regionally recurrent breast cancer, characterised in that the cancer has recurred or progressed following at least one prior line of endocrine therapy, and wherein the medicament for use delivers:
a) an improvement in the median time to disease progression of at least 3.5 months relative to that observed with fulvestrant treatment; and/or
b) a hazard ratio for use of camizestrant treatment relative to fulvestrant treatment which is less than or equal to 0.67; and/or
c) a median time to disease progression of at least 7 months.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the cancer has recurred or progressed following at least one prior line of endocrine therapy.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy, and:
a) the use delivers an improvement to the median time to disease progression of at least 3.5 months more than that observed with fulvestrant treatment; and/or
b) the hazard ratio for ngSERD treatment relative to fulvestrant treatment is less than or equal to 0.67; and/or
c) the use delivers a median time to disease progression of at least 7 months.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy and at least one prior line of therapy with a CDK4/6 inhibitor.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy and at least one prior line of therapy with a CDK4/6 inhibitor, and:
a) the use delivers an improvement to the median time to disease progression of at least 1.7 months more than that observed with fulvestrant treatment; and/or
b) the hazard ratio for ngSERD treatment relative to fulvestrant treatment is less than or equal to 0.68; and/or
c) the use delivers a median time to disease progression of at least 3.8 months.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy and the cancer has been identified as having visceral metastases.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy and the cancer has been identified as having visceral metastases, and:
a) the use delivers an improvement to the median time to disease progression of at least 3.6 months more than that observed with fulvestrant treatment; and/or
b) the hazard ratio for ngSERD treatment relative to fulvestrant treatment is less than or equal to 0.55; and/or
c) the use delivers a median time to disease progression of at least 5.6 months.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy, and wherein the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G).

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy, and wherein the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G) on the basis of a test of a sample obtained from the patient (for example a test performed by analysing circulating tumor DNA), for example wherein the sample is a tumor biopsy or a blood sample.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the breast cancer has recurred or progressed following at least one prior line of endocrine therapy and the use improves overall survival relative to treatment with fulvestrant.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the cancer has recurred or progressed following at least one prior line of endocrine therapy, wherein:
a) the use delivers an improvement to the median time to disease progression of at least 3.5 months relative to that observed with fulvestrant treatment; and/or
b) the hazard ratio for use of camizestrant treatment relative to fulvestrant treatment is less than or equal to 0.67; and/or
c) the use delivers a median time to disease progression of at least 7 months.

In one embodiment there is provided a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy.

In one embodiment there is provided a kit comprising a medicament containing an ngSERD and instructions for use of the pharmaceutical composition in the treatment of HR+/HER2− metastatic or loco-regionally recurrent breast cancer, characterised in that the cancer has recurred or progressed following at least one prior line of endocrine therapy, wherein use of the kit delivers:
a) the median time to disease progression is at least 3.5 months more than that observed with fulvestrant treatment; and/or
b) the hazard ratio for ngSERD treatment relative to fulvestrant treatment is less than or equal to 0.67; and/or
c) the median time to disease progression is at least 7 months.

In one embodiment there is provided a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy and at least one prior line of therapy with a CDK4/6 inhibitor.

In one embodiment there is provided a kit comprising a medicament containing an ngSERD and instructions for use of the pharmaceutical composition in the treatment of HR+/HER2− metastatic or loco-regionally recurrent breast cancer, characterised in that the cancer has recurred or progressed following at least one prior line of endocrine therapy, wherein use of the kit delivers:
a) the median time to disease progression is at least 1.7 months more than that observed with fulvestrant treatment; and/or
b) the hazard ratio for ngSERD treatment relative to fulvestrant treatment is less than or equal to 0.68; and/or
c) the median time to disease progression is at least 3.8 months.

In one embodiment there is provided a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy and the cancer has been identified as having visceral metastases.

In one embodiment there is provided a kit comprising a medicament containing an ngSERD and instructions for use of the pharmaceutical composition in the treatment of HR+/HER2− metastatic or loco-regionally recurrent breast cancer, characterised in that the cancer has recurred or progressed following at least one prior line of endocrine therapy, wherein use of the kit delivers:
a) the median time to disease progression is at least 3.6 months more than that observed with fulvestrant treatment; and/or
b) the hazard ratio for ngSERD treatment relative to fulvestrant treatment is less than or equal to 0.55; and/or
c) the median time to disease progression is at least 5.6 months.

In one embodiment there is provided a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy and the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G).

In one embodiment there is provided a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy and the cancer has been identified as having a mutation of the estrogen receptor (for example a mutation to the estrogen receptor selected from E380Q, V422del, S463P, L536H, L536P, L536R, Y537C, Y537D, Y537N, Y537S and D538G) on the basis of a test of a sample obtained from the patient (for example a test performed by analysing circulating tumor DNA), for example wherein the sample is a tumor biopsy or a blood sample.

In one embodiment there is provided a kit comprising a medicament containing an ngSERD and instructions for use of the pharmaceutical composition in the treatment of HR+/HER2− metastatic or loco-regionally recurrent breast cancer in a patient whose cancer has recurred or progressed following at least one prior line of endocrine therapy and the use improves overall survival relative to treatment with fulvestrant.

In one embodiment there is provided a pharmaceutical composition comprising a ngSERD (for example camizestrant or a pharmaceutically acceptable salt thereof which is optionally orally administered once daily at a dose of 75 mg or 150 mg) and at least one pharmaceutically acceptable excipient for use in the treatment of HR+, human epidermal growth factor receptor 2 negative HER2−, metastatic or loco-regionally recurrent breast cancer, characterised in that the cancer has recurred or progressed following at least one prior line of endocrine therapy, wherein use of the kit delivers:
a) the use delivers an improvement the median time to disease progression of 3.5 months relative to that observed with fulvestrant treatment; and/or
b) the hazard ratio for use of camizestrant treatment relative to fulvestrant treatment is less than or equal to 0.67; and/or
c) the use delivers a median time to disease progression of at least 7 months.

In one embodiment there is provided a kit comprising a pharmaceutical composition comprising a camizestrant and at least one pharmaceutically acceptable excipient and instructions for use of the pharmaceutical composition in the treatment of HR+, HER2−, metastatic or loco-regionally recurrent breast cancer, characterised in that the cancer has recurred or progressed following at least one prior line of endocrine therapy, wherein use of the kit delivers:
a) an improvement in the median time to disease progression of 3.5 months relative to that observed with fulvestrant treatment; and/or
b) a hazard ratio for use of camizestrant treatment relative to fulvestrant treatment is less than or equal to 0.67; and/or
c) a median time to disease progression of at least 7 months.

Further details on the SERENA-2 clinical trial protocol are provided below. The trial was initiated with 3 doses of camizestrant (AZD9833), namely 75 mg, 150 mg and 300 mg, albeit only 20 patients were recruited on to the 300 mg arm before this arm was discontinued. Accordingly, no results are provided for the 300 mg arm.

### EXAMPLES

Abbreviations. B=blood; BoR=Best objective response; CA15-3=cancer antigen 15-3; BICR=Blinded Independent Central Review; CBR24=clinical benefit rate at 24 weeks; CSP=Clinical Study Protocol; CTC=circulating tumour cell; ctDNA=circulating tumour DNA; Cx=Cycle x; Dx=Day x; DoR=duration of response; ECG=electrocardiogram; ECOG=Eastern Cooperative Oncology Group; EORTC=European Organisation for Research and Treatment of Cancer; EORTC QLQ BR23=EORTC quality of life questionnaire—breast cancer module; EORTC QLQ C30=EORTC quality of life questionnaire—core questionnaire, EOT=end of treatment; EQ 5D 5L=EuroQol 5 Dimension 5 level; HRQOL=health-related quality of life; ICF=informed consent form; IM=intramuscular; ITT=intention-to-treat; NEI VFQ-25=National Eye Institute 25-Item Visual Function Questionnaire; ORR=objective response rate; OS=overall survival; PFS=progression-free survival; PGI-BR=patient global impression of benefit-risk; PGIC=patient global impression of change; PGIS=patient global impression of severity; PGI TT=patient global impression of treatment tolerability; PgR=progesterone receptor; PK=pharmacokinetics; PO=oral (per os); PRO=patient-reported outcome; RECIST=Response Evaluation Criteria in Solid Tumours; SAE=Serious adverse event; SRC=Safety review committee; U=urine; ULN=upper limit of normal; WHO=World Health Organisation.

### Summary

A randomised, open-label, parallel-group, multicentre phase 2 study comparing the efficacy and safety of oral AZD9833 versus fulvestrant in women with advanced er-positive her2-negative breast cancer was carried out according to the following protocol and schedule of activities. The study evaluated the efficacy and safety of AZD9833 (75, 150 and 300 mg, oral [PO]) administered once daily as a monotherapy in comparison with fulvestrant administered according to its label (i. e., slow IM injection into the buttocks [1 to 2 minutes per injection] as two 5-mL injections, 1 in each buttock, on Day 1, Day 15, Day 29 and 4-weekly thereafter).

TABLE 6
Schedule of Activities

| Screening | Treatment period (28-day cycles) | | | | | | | | | | | |
|---|---|---|---|---|---|---|---|---|---|---|---|---|
| Visit | | | | | | | | | | | | |
| 1 | 2 | 3 | 4 | 5 | 7 | 8 | 9 | 11 | ≥12 | | | |
| Cycle | | | | | | | | | | | | |
| | C1 | | | C2 | C3 | C4 | C5 | C6 | C ≥ 7 | Post-treatment period | | |
| | | | | | | | | | | | 28-day | |
| Cycle day | | | | | | | | | | EOT | safety | |
| — | D 1 | D 8 | D 15 | D 1 | D 1 | D 1 | D 1 | D 1 | D 1 | visit | follow-up | |
| Study day | | | | | | | | | | | | Survival |
| −28 to −1 | 1 | 8 | 15 | 29 | 57 | 85 | 113 | 141 | ≥169 | EOT | EOT + 28 | follow-up |
| Week (Day 1 of) | | | | | | | | | | | | 12- |
| −4 to −1 | 1 | 2 | 3 | 5 | 9 | 13 | 17 | 21 | ≥25 | EOT | EOT + 4 | weekly |

Visit window (days)

| | | | | | | | | | | |
|---|---|---|---|---|---|---|---|---|---|---|
| ±1 | ±1 | ±3 | ±3 | ±3 | ±3 | ±3 | ±3 | +7 | +7 | ±14 |

| | |
|---|---|
| Informed consent | X |
| Eligibility criteria | X |

Routine clinical procedures

| Demography, | X | | | | | | | | | | |
|---|---|---|---|---|---|---|---|---|---|---|---|
| medical/surgical history, | | | | | | | | | | | |
| baseline characteristics | | | | | | | | | | | |
| Concomitant medication | X | At every visit and may be conducted by phone | | | | | | | | | |
| Survival status, anti- | | | | | | | | | | | X |
| cancer treatments | | | | | | | | | | | |
| ECOG/WHO | X | X | X | X | X | X | X | every | X | X | |
| performance status | | | | | | | | 2 cycles | | | |

Routine safety assessments

| Adverse events | X | At every visit and may be conducted by phone | | | | | | | | | | |
|---|---|---|---|---|---|---|---|---|---|---|---|---|
| Physical examination | X | X | | | X | X | X | X | X | every | X | X |
| | | | | | | | | | | 2 cycles | | |
| Weight, height ^{a} | X | X | | | X | X | X | X | X | every | X | X |
| | | | | | | | | | | cycle | | |
| Vital signs (temperature, | X | X | X | X | X | X | X | X | X | every | X | X |
| pulse rate, blood | | | | | | | | | | 2 cycles | | |
| pressure) | | | | | | | | | | | | |
| Triplicate ECGs | X | pre- | X | X | X | X | X | X | X | every | X | X |
| | | dose | | | | | | | | 2 cycles | | |
| 24-hour ECG | X | | | Once between | | | | | | | | |
| | | | | D 15 and D 29 | | | | | | | | |
| Echocardiogram^{g} | X | | | | X | | | every | | | | X |
| | | | | | | | | 3rd | | | | |
| | | | | | | | | cycle | | | | |
| Clinical chemistry, | X | X | | X | X | X | X | X | X | every | X | |
| haematology, urinalysis | | | | | | | | | | 2 cycles | | |
| Coagulation tests | X | X | | | X | | | | | | | |

PK sampling for AZD9833 and, if appropriate, metabolite(s) ^{b}

| | | |
|---|---|---|
| PK sample pre-dose | X | X |
| PK samples post-dose (2, | X | |
| 4 hours) | | |

Efficacy assessments

| | | | | |
|---|---|---|---|---|
| Tumour imaging | X | X ^{c} | X ^{c} | 8- |
| (RECIST version 1.1) | | | | weekly ^{c} |
| Bone scan, skeletal | X ^{d} | | | |
| survey | | | | |

Biomarker analyses

| | | | | | | | | | |
|---|---|---|---|---|---|---|---|---|---|
| Blood sample for ctDNA | X | pre- | X | X | X | X | X | 8- | X |
| | | dose | | | | | | weekly ^{e} | |
| Blood sample for CA15-3 | X | pre- | X | X | X | X | X | 8 | X |
| | | dose | | | | | | weekly ^{e} | |
| Blood sample for CTC | X | pre- | | X | | X | | | X |
| | | dose | | | | | | | |
| Archival tumour tissue | X | | | | | | | | |
| Optional tumour biopsy | X | | | X | | | | at | |
| (for selected patients) | | | | | | | | progression | |

Patient reported outcomes

| | | | | | | | |
|---|---|---|---|---|---|---|---|
| HRQoL questionnaires | | X | X | X | 8- | X | X |
| (EORTC QLQ-C30, | | | | | weekly ^{e} | | |
| EORTC QLQ-BR23, | | | | | | | |
| NEI VFQ-25, | | | | | | | |
| EQ-5D-5L, PGIS, PGIC, | | | | | | | |
| PGI-TT, PGI-BR) | | | | | | | |
| PRO interviews ^{f} | X | | X | X | | | |

Optional genetic sampling

| | |
|---|---|
| Blood sample | X |

Study treatment

| | | | | | | | | | |
|---|---|---|---|---|---|---|---|---|---|
| Randomisation | X | | | | | | | | |
| Fulvestrant (500 mg IM) | X | X | X | X | X | X | X | every | |
| | | | | | | | | cycle | |
| Dispense/collect | X | | X | X | X | X | X | every | X |
| AZD9833 | | | | | | | | cycle | |

| | |
|---|---|
| AZD9833 (PO) | 75 mg, 150 mg, or 300 mg once daily |

^{a} Height will be measured at screening only.
^{b} Pharmacokinetic samples will only be collected for patients treated with AZD9833.
^{c} The time window for tumour imaging is ±7 days. Assessments will be conducted until disease progression.
^{d} The bone scan/skeletal survey should be performed within 12 weeks of treatment start (C1D1).
^{e} Sample collection and completion of HRQoL questionnaires will be done 8-weekly (D 1 of every 2^{nd} cycle) from Week 25 (C7) to coincide with tumour RECIST assessment.
^{f} PRO interviews will only be conducted for patients enrolled in the United States, United Kingdom, and Spain. The interviews will be conducted by phone. The 1^{st} interview will occur at baseline (prior to 1^{st} dose within screening period). Vendor conducting interviews will be notified within a day of identification of an eligible patient and the date of scheduled C1D1. Best efforts will be used to schedule all interviews within the windows allowed. The 2^{nd} interview will occur 4 weeks (±7 days) from C1D1 and the 3^{rd} interview will occur 12 weeks (±21 days) from C1D1.
^{g}Echocardiograms time window for visits C2D1, C5D1 and every 3rd cycle thereafter, and 28-day follow-up echocardiograms is ±7 days

TABLE 7
Objectives and Endpoints

| Primary Objective: | Endpoint/Variable: |
|---|---|
| To determine the clinical efficacy (as assessed by PFS) of | PFS assessed by the Investigator as defined by RECIST |
| AZD9833 when compared to fulvestrant in women with | version 1.1 |
| advanced ER-positive HER2-negative breast cancer | |
| Secondary Objectives: | Endpoints/Variables: |
| To determine anti-tumour effect of AZD9833 when | Based on tumour response assessed by the Investigator, as |
| compared to fulvestrant in women with advanced | defined by RECIST version 1.1: |
| ER-positive HER2-negative breast cancer | ORR |
| | DoR |
| | Best percentage change in tumour size and |
| | percentage change in tumour size at 16 weeks |
| To determine the effect of AZD9833 on survival and clinical | OS |
| benefit when compared to fulvestrant in women with | CBR₂₄ |
| advanced ER-positive HER2-negative breast cancer | |
| To evaluate the PK of AZD9833 in this patient population | Plasma concentrations of AZD9833 and, if appropriate, |
| at steady state | metabolite(s) on Day 15 (pre- and post-dose) and Day 29 |
| | (pre-dose) |
| To evaluate the pharmacodynamics of AZD9833 and | Percent change from baseline in ER and PgR |
| fulvestrant in a subgroup of patients with advanced | expression assessed by the manual H-score |
| ER-positive HER2-negative breast cancer | method. |
| | Percent change from baseline in Ki67 labelling |
| | index |
| To evaluate the effect of AZD9833 and fulvestrant on the | Changes from baseline in total/subscale scores of the |
| patients' HRQoL, as assessed by patient-completed HRQoL | EORTC QLQ-C30, EORTC QLQ-BR23, NEI VFQ-25, and EQ- |
| questionnaires | 5D-5L |
| Safety Objective: | Endpoints/Variables: |
| To evaluate the safety and tolerability of AZD9833 when | AEs/SAEs |
| compared to fulvestrant in women with advanced | Vital signs, ECGs, clinical chemistry, |
| ER-positive HER2-negative breast cancer | haematology, urinalysis parameters |
| Exploratory Objectives | Endpoints/Variables: |
| To investigate predictive markers of response and/or | Change from baseline in amount and mutation |
| acquired resistance to AZD9833 and fulvestrant | status of cancer-associated genes in ctDNA, CTC, |
| | and/or tumour samples |
| To assess the impact of tumour mutation status on | Subgroup analysis of tumour response (PFS, ORR, |
| response to treatment and acquired resistance | DoR, percent change in tumour size) by tumour |
| | mutation status |
| | Change in tumour mutational status during |
| | treatment |
| To evaluate the effect of AZD9833 and fulvestrant on the | Spontaneously and probed reported |
| patients' HRQoL, as assessed by a 1-to-1 telephone | symptoms/impacts |
| interview | Ranking of most bothersome symptoms/impacts |
| | Reported disturbance rating of each |
| | symptom/impact |
| To examine overall symptoms, change in condition, | PGIS, PGIC, PGI-TT, and PGI-BR |
| tolerability and patient-perceived benefit/risk | |
| Optional genotyping: To collect and store DNA according | Results from future exploratory research may be reported |
| to each country's local and ethical procedures for future | outside of the CSR. |
| exploratory research into genes/genetic variation that may | |
| influence response to treatment | |

### Overall Study Design

This is a randomised, open-label, parallel-group, multicentre Phase 2 study to compare the efficacy and safety of daily PO AZD9833 versus IM fulvestrant in women with advanced ER positive HER2 negative breast cancer. Post-menopausal women with histologically or cytologically confirmed metastatic or loco-regionally recurrent disease before randomisation and fulfilling all of the inclusion criteria and none of the exclusion criteria will be included. Randomisation will be stratified according to the prior use of CDK4/6 inhibitors and the presence of liver and/or lung metastases.

After the screening visit and confirmation of eligibility, patients will be randomly assigned in a 1:1:1:1 ratio to receive 1 of the following 4 treatments, consisting in 4-week treatment cycles until disease progression (assessed by the Investigator as defined by Response Evaluation Criteria in Solid Tumours [RECIST] version 1.1):
AZD9833 (75 mg, PO, once daily)
AZD9833 (150 mg, PO, once daily)
AZD9833 (300 mg, PO, once daily)
Fulvestrant (500 mg IM, Day 1, Day 15, Day 29, and 4-weekly thereafter)

During the treatment period, patients will attend visits on:
Day 1, Day 8, and Day 15 of Cycle 1
Day 1 of each subsequent cycle until treatment discontinuation

After the treatment period, patients will attend 2 safety follow-up visits (at the time of treatment discontinuation and 28 days later) and will continue to be followed for survival. Throughout the study, patients will be asked to report adverse events (AEs) and the use of concomitant medication.

Safety assessments (physical examination, vital signs, electrocardiograms [ECGs], clinical safety laboratory assessments) will be performed at screening, on Day 1 of every cycle (up to Cycle 6) and every 2 cycles (from Cycle 6) and at the end of treatment (EOT) visit. Echocardiograms will be performed at screening, on Day 1 of cycle 2 and 5, every 3 cycles thereafter and at the 28 day follow up visit. Safety assessments will also be performed on Cycle 1 Day 8 (vital signs and triplicate ECGs), Cycle 1 Day 15 (vital signs, ECGs, clinical chemistry, haematology, and urinalysis), at the 28 day safety follow-up (physical exam and vital signs).

Tumour imaging will be performed for the assessment tumour response according to RECIST version 1.1 at screening and every 8 weeks from Week 9 until disease progression.

Patients will complete health-related quality of life (HRQOL) questionnaires on Day 1 of Cycles 1, 2, and 4, then 8-weekly (Day 1 of every 2nd cycle) from Week 25 (Cycle 7) to coincide with tumour imaging, at the EOT and/or disease progression, and at the 28 day safety follow-up.

Blood and plasma samples for circulating tumour (ctDNA) and cancer antigen 15-3 (CA15-3) analyses will be collected at screening, on Day 1 and Day 15 of Cycle 1, on Day 1 of Cycles 2 to 5, 8-weekly (Day 1 of every 2nd cycle) from Week 25 (Cycle 7) to coincide with tumour imaging, at the EOT and/or disease progression.

Blood samples for circulating tumour cells (CTCs) will be collected at screening, on Day 1 of Cycles 1, 2, and 4, and at the EOT.

For patients receiving AZD9833, blood samples will be collected for pharmacokinetic (PK) assessments on Cycle 1 Day 15 and Cycle 2 Day 1.

For patients who provide specific consent, the following optional assessments will be performed:
Up to 12 patients per treatment group will be selected such that they are suitable for providing 1 pre-treatment and 1 on-treatment paired tumour biopsy sample. If provision of paired biopsies becomes clinically unfeasible for a selected patient during the course of her care, the patient may be replaced until up to 12 evaluable biopsy pairs are collected within each treatment group.
An optional blood sample will be collected at pre-dose on Day 1 of Cycle 1 or later during the study for future genetic research.

Number of patients. This study will screen approximately 360 patients (assuming a screen failure rate of 20%), in order to randomise 288 patients into 4 treatment groups in a 1:1:1:1 ratio:
AZD9833 75 mg: 72 patients
AZD9833 150 mg: 72 patients
AZD9833 300 mg: 72 patients (NB recruitment for this arm ceased after 20 patients)
Fulvestrant: 72 patients

Treatments and treatment duration. Patients will receive study treatment until objective disease progression (according to RECIST version 1.1) or other discontinuation criteria are met.

AZD9833 will be administered once daily:
75 mg: 3×25-mg tablets.
150 mg: 1×100-mg tablet+2×25-mg tablets.
300 mg: 3×100-mg tablets.

Fulvestrant will be administered on Day 1, Day 15 (±1 day), Day 29 (±3 days), and 4-weekly thereafter:
500 mg: 2×5-ml IM injections.

Study treatments are summarised in Table 8.

TABLE 8
Study Treatments

| | Treatment 1 | Treatment 2 | Treatment 3 | Treatment 4 |
|---|---|---|---|---|
| Treatment name | AZD9833 | | | Fulvestrant |
| Dosage | 25-mg and | | | 250 mg/5 ml solution |
| formulation | 100-mg tablets | | | for IM injection |
| Dosage | 75 mg | 150 mg | 300 mg | 500 mg |
| level(s) | once | once | once | Day 1, Day 15, Day 29, and |
| | daily | daily | daily | 4-weekly thereafter |
| Route of | PO | | | IM injection |
| administration | | | | |
| Dosing | 3 × 25-mg | 2 × 25-mg tablets + | 3 × 100-mg | 2 consecutive 250-mg injections |
| instructions | tablets | 1 × 100-mg tablet | tablets | from 2 prefilled syringes |

| | AZD9833 should be taken in the morning, with or without | (1 in each buttock) |
|---|---|---|
| | food, at approximately the same time. | |
| Packaging and | AZD9833 will be provided in bottles. Each bottle will be | Fulvestrant will be packed into |
| labelling | labelled in accordance with country regulatory | single-dose cartons containing |
| | requirements. | 2 labelled prefilled syringes. Each |
| | | carton will be labelled in |
| | | accordance with country |
| | | regulatory requirements. |
| Provider | AstraZeneca | Commercial fulvestrant |

(Faslodex) will be provided by
AstraZeneca.

Study population. Prospective approval of protocol deviations to inclusion/exclusion criteria, also known as protocol waivers or exemptions, is not permitted. Each patient should meet all of the inclusion criteria and none of the exclusion criteria for this study in order to assigned/randomised to a study intervention. Patients who are enrolled and do not meet the entry requirements are screen failures. In this protocol, “enrolled” patients are defined as those who sign informed consent. “Randomised” patients are defined as those who undergo randomisation and receive a randomisation number. Patients are eligible to be included in the study only if all of the following inclusion criteria and none of the exclusion criteria apply.

Inclusion criteria. Informed consent. Provision of signed and dated, written informed consent prior to any mandatory study specific procedures, sampling, and analyses. Patients are also required to consent to the provision of archival tumour biopsies. For patients who consent, provision of signed and dated written genetic informed consent prior to collection of samples for genetic analysis.

Age and gender. Female patients aged at least 18 years.

Menopausal status. Post-menopausal defined as meeting at least 1 of the following criteria:
Have undergone a bilateral oophorectomy.
Age ≥60 years.
Age ≥50 years and with cessation of regular menses ≥12 months and with an intact uterus in the absence of gonadotropin-releasing hormone (GnRH) agonist, oral contraception or hormone replacement therapy.
Age <50 years and with cessation of regular menses ≥12 months and follicle stimulating hormone (FSH) and oestradiol levels in the post-menopausal range (utilising ranges from the local laboratory facility) and with an intact uterus in the absence of gonadotropin-releasing hormone (GnRH) agonist, oral contraception or hormone replacement therapy.

Disease characteristics. Histologically or cytological confirmation of adenocarcinoma of the breast. Documented ER-positive status of primary or metastatic tumour tissue, according to the local laboratory parameters and where those laboratory parameters are in accordance with accepted diagnostic guidelines (e. g., American Society of Clinical Oncology/College of American Pathologists Guideline Recommendations for Immunohistochemical Testing of Estrogen and Progesterone Receptors in Breast Cancer, [Hammond et al. 2010]).

Documented HER2-negative status defined as an immunohistochemistry (IHC) Score 0 or 1+ or negative by in situ hybridisation (ISH; FISH/CISH/SISH); if IHC 2+, ISH negativity is required. Where available, assessment of ER and HER2 status should be based on the most recent tumour biopsy sample. according to the local laboratory parameters and where those laboratory parameters are in accordance with accepted diagnostic guidelines (e. g., American Society of Clinical Oncology/College of American Pathologists Guideline Recommendations for Immunohistochemical Testing of Estrogen and Progesterone Receptors in Breast Cancer, [Hammond et al. 2010]).

Metastatic disease or loco-regionally recurrent disease suitable for treatment with fulvestrant.

Radiological or other objective evidence of progression on or after the last systemic therapy prior to starting study treatment. Tumour marker progression alone is not considered objective evidence of progression.

Patients must have:
at least 1 lesion, not previously irradiated, that can be measured accurately at baseline as ≥10 mm in the longest diameter (except lymph nodes which must have short axis ≥15 mm) with computed tomography (CT) or magnetic resonance imaging (MRI) which is suitable for accurate repeated measurements, or
in absence of measurable disease as defined above, at least 1 lytic or mixed (lytic+sclerotic) bone lesion that is amenable to serial assessment by CT or MRI; patients with sclerotic/osteoblastic bone lesions only in the absence of measurable disease are not eligible.

Eastern Cooperative Oncology Group (ECOG)/World Health Organisation (WHO) performance status 0 to 1, with no deterioration over the previous 2 weeks and a minimum life expectancy of 12 weeks.

Prior chemotherapy, endocrine therapy and other anti-cancer therapy. Prior endocrine therapy as follows:
Recurrence or progression on at least one line of endocrine therapy.
No more than 1 line of endocrine therapy for advanced disease.
No more than 1 line of chemotherapy for advanced disease. A chemotherapy line in advanced disease is an anti-cancer regimen(s) that contains at least one cytotoxic chemotherapy agent and given for 21 days or longer. If a cytotoxic chemotherapy regimen was discontinued for a reason other than disease progression and lasted less than 21 days, then this regimen does not count as a prior line of chemotherapy. Repeat administration of the same anti-cancer regimen on a separate occasion does not count as a new line of chemotherapy.
Prior treatment with CDK4/6 inhibitors is permitted.
No prior treatment with fulvestrant, oral SERD, or related therapies (e.g., ER, proteolysis targeting chimeras [PROTACs], selective ER covalent antagonists [SERCAs] in the metastatic setting).

Inclusion criteria for the paired tumour biopsy. Disease suitable for paired baseline and on-treatment tumour biopsies. Washout from research subgroup. prior tamoxifen: 4 months to elapse from last tamoxifen dose to pre-dose on-study biopsy. Provision of signed, written, and dated informed consent for tumour biopsies.

Exclusion criteria. Patients must not enter the study if any of the following exclusion criteria are fulfilled.

Prior/concomitant therapy. Intervention with any of the following:
Any cytotoxic chemotherapy, investigational agents or other anti-cancer drugs for the treatment of breast cancer from a previous treatment regimen or clinical study within 14 days of the first dose of study treatment.
Use of systemic oestrogen-containing hormone replacement therapy within 6 months prior to the first dose of study treatment.

Medications or herbal supplements known to be strong inhibitors/inducers of cytochrome P450 (CYP) 3A4/5, sensitive CYP2B6 substrates, and drugs which are substrates of CYP2C9 and/or CYP2C19 which have a narrow therapeutic index i.e., warfarin (and other coumarin-derived vitamin K antagonist anticoagulants) and phenytoin or inability to stop use within the washout period as specified in Appendix B prior to receiving the first dose of study treatment.
Drugs that are known to prolong QT and have a known risk of torsades de pointes, as indicated in Appendix B 1.
Radiotherapy with a limited field of radiation for palliation within 1 week of the first dose of study treatment, and/or radiation to more than 30% of the bone marrow or a wide field of radiation within 4 weeks of the first dose of study treatment.
Major surgical procedure or significant traumatic injury, as judged by the Investigator, within 4 weeks of the first dose of study treatment, or an anticipated need for major surgery and/or any surgery requiring general anaesthesia during the study.

Medical conditions. Any unresolved toxicities from prior therapy greater than CTCAE Grade 1 at the time of starting study treatment, with the exception of alopecia and chemotherapy-related peripheral neuropathy.

Presence of life-threatening metastatic visceral disease or uncontrolled CNS metastatic disease as judged by the Investigator. Patients with spinal cord compression and/or brain metastases may be enrolled if definitively treated (e.g., surgery or radiotherapy) and stable off steroids for at least 4 weeks prior to start of study treatment.

Any of the following criteria:
Any evidence of severe or uncontrolled systemic diseases, including uncontrolled hypertension and active bleeding diatheses, or e.g., infection requiring intravenous antibiotic therapy, which in the Investigator's opinion makes it undesirable for the patient to participate in the study or which would jeopardise compliance with the protocol,
Immunocompromised patients, e.g., patients who are known to be serologically positive for human immunodeficiency virus (HIV).
Patients with known active hepatitis (i.e., hepatitis B or C)

Any of the following cardiovascular criteria:
Mean resting QTcF >470 msec obtained from screening triplicate ECG.
Resting heart rate <45 bpm
Any clinically important abnormalities in rhythm, conduction, or morphology of resting ECG (e.g., complete left bundle branch block, second- and third-degree heart block), or clinically significant sinus pause, or sick sinus syndrome. Patients with controlled atrial fibrillation can be enrolled.
Any factors that increase the risk of QTc prolongation or risk of arrhythmic events such as symptomatic heart failure, congenital long QT syndrome, immediate family history of long QT syndrome, or unexplained sudden death at <40 years of age; hypertrophic cardiomyopathy and clinically significant stenotic valve disease; clinically significant hypokalaemia, hyperkalemia, hypo- and hyper magnesaemia, hypo- and hyper-calcaemia.
Left ventricular ejection fraction <50%, and/or experience of any of the following procedures or conditions in the preceding 6 months: coronary artery bypass graft, angioplasty, vascular stent, myocardial infarction, unstable angina pectoris, congestive heart failure New York Heart Association (NYHA) Grade ≥2, cerebrovascular accident, or transient ischaemic attack.
Uncontrolled hypertension. Hypertensive patients may be eligible, but blood pressure must be adequately controlled at baseline. Patients may be rescreened regarding the blood pressure requirement.
Symptomatic hypotension

Inadequate bone marrow reserve or organ function as demonstrated by any of the following laboratory values:
Absolute neutrophil count (ANC) <1.5×109/L
Platelet count <100×109/L
Haemoglobin (Hb) <90 g/L
Alanine aminotransferase (ALT) >2.5×the upper limit of normal (ULN)
Aspartate aminotransferase (AST) >2.5×ULN
Total bilirubin (TBL) >1.5×ULN or >3×ULN in the presence of documented Gilbert's Syndrome (unconjugated hyperbilirubinaemia)
Estimated glomerular filtration rate (eGFR) <50 mL/min
Refractory nausea and vomiting, uncontrolled chronic gastrointestinal diseases, inability to swallow the formulated product, or previous significant bowel resection that would preclude adequate absorption of AZD9833.

Other exclusions:
History of hypersensitivity to active or inactive excipients of AZD9833 or fulvestrant.
Involvement in the planning and conduct of the study (applies to both AstraZeneca staff and/or staff at the study site).
Previous randomisation in the present study.
Judgement by the Investigator that the patient should not participate in the study if the patient is unlikely to comply with study procedures, restrictions and requirements.
Male patients are excluded from this study.
Women of childbearing potential are excluded from this study.

Lifestyle restrictions. Patients should abstain from eating large amounts of grapefruit and Seville oranges (and other products containing these fruits [e.g., grapefruit juice or marmalade]) during the study (e.g., no more than a small glass of grapefruit juice [120 mL], half a grapefruit, or 1 to 2 teaspoons [15 g] of Seville orange marmalade daily). There are no food restrictions for AZD9833 (i.e., AZD9833 may be taken with or without food). There are no food restrictions for fulvestrant.

Measures to minimise bias: randomisation and blinding. This is an open-label study. All patients will receive active treatment. As the Investigators will not be blinded to study treatments, a BICR of tumour scans will be performed to provide independent tumour response assessment in addition to response evaluation by Investigator. To reduce potential bias during the study, eligible patients will be randomly allocated at a 1:1:1:1 ratio to receive 1 of the 4 study treatments.

Patients will be randomised as they become eligible. Once the eligibility of a patient has been confirmed, the Investigator (or designee) will notify the centralised IWRS. Randomisation should take place as close to the start of study treatment as possible, with a maximum delay of 3 days. The randomisation will be stratified based on 2 stratification factors:
prior use of CDK4/6 inhibitors in any setting (yes/no)
presence of lung and/or liver metastases (yes/no)

The 1^{st} factor (i.e., prior use of CDK4/6 inhibitors) will be controlled such that between 32 and up to a maximum of 40 patients per treatment arm (out of 72 planned patients) are included in each stratum, to ensure approximately 50% of patients in each treatment group at the final analysis will be naïve to CDK4/6 inhibitors. The cap will allow a maximum number of 40 patients to be enrolled per stratum, but will not exceed the total planned per treatment group. There will be no cap imposed on the 2^{nd} stratification factor (i.e., presence of lung and/or liver metastases).

Efficacy assessments. Assessment of tumour response. The assessment of tumour response per RECIST version 1.1 will be used to determine the clinical activity (assessed by PFS) and anti-tumour activity (assessed by ORR, clinical benefit rate [CBR]) of AZD9833 and fulvestrant.

Tumour imaging. Tumour imaging should be performed per the SoA (Table 6). Baseline tumour assessments should encompass all areas of known predilection for metastases in the disease under evaluation and should additionally investigate areas that may be involved based on signs and symptoms of individual patients. Baseline assessments should be performed no more than 28 days before the start of study treatment, ideally, as close as possible to the start of study treatment.

If an unscheduled assessment is performed, and the patient has not progressed, every attempt should be made to perform the subsequent assessments at their scheduled visits. This schedule is to be followed in order to minimise any unintentional bias caused by some patients being assessed at a different frequency than other patients.

Investigator's assessment of tumour response per RECIST version 1.1. Tumour response will be assessed by the Investigators per RECIST version 1.1 (Eisenhauer et al. 2009). From the Investigator's review of the imaging scans, the evaluation of TLs, NTLs and new lesions will be used to determine the overall visit response for each patient. Overall visit responses for each visit will then be used to determine if and when a patient has progressed in accordance with RECIST and their best objective response to study treatment.

Blinded Independent Central Review of tumour assessments. Coded copies of all imaging assessments (regardless of modality and including unscheduled visit scans) will be sent to an appointed Contract Research Organisation (CRO) for central analysis. Results of this independent review will not be communicated to Investigators, and the management of patients will be based solely upon the results of the RECIST assessment conducted by the Investigator. The imaging scans will be reviewed by 2 independent radiologists using RECIST version 1.1 and will be adjudicated, if required (i.e., 2 reviewers will review the scans and, in case of a disagreement, adjudication is performed by a separate reviewer). The independent reviewers will be blinded to study treatment.

The BICR will be conducted on an ongoing basis throughout the study. Where possible scans will be batched, and an in-patient series of assessments read together. For each patient, the BICR will define the overall visit response (i.e., the response obtained overall at each visit by assessing TLs, NTLs, and new lesions) and no programmatic derivation of overall visit response is necessary.

Bone scan or skeletal survey. All patients should have a baseline bone scan or skeletal survey performed no more than 12 weeks before and as close as possible to the start of study treatments. Additional on-study bone scans or skeletal surveys may be performed, if clinically indicated. Bone lesions identified on an isotopic bone scan at baseline and confirmed by CT, MRI, or X ray should be recorded as NTLs and followed by the same method (CT, MRI, or X-ray), as indicated in the SoA (Table 6).

Survival follow-up. After discontinuation of study treatment, the survival status of the patients will continue to be followed every 12 weeks via phone until closure of the clinical study database. Any new anti-cancer therapies commenced by the patient during the survival follow-up period will be documented in the clinical database. A survival follow-up phone call will be made in the week following the DCO date for each survival analysis.

Post Primary Analysis. At the time of final analysis, the clinical study database will close to new data. Patients are, however, permitted to continue to receive study treatment beyond the closure of the database if, in the opinion of the Investigator, they are continuing to receive benefit from study treatment.

Clinical outcome assessments. Performance status. Performance status will be assessed at the visits indicated in the SoA (Table 6), according to ECOG/WHO criteria as follows:
Fully active, able to carry on all pre-disease activities without restrictions.
Restricted in physically strenuous activity but ambulatory and able to carry out work of a light or sedentary nature (e.g., light housework, office work).
Ambulatory and capable of self-care but unable to carry out any work activities. Up and about more than 50% of waking hours.
Capable of only limited self-care, confined to bed or chair more than 50% of waking hours.
Completely disabled. Cannot carry on any self-care. Totally confined to bed or chair.

Health-related quality of life questionnaires. PRO measures will be used to examine the impact of treatment on symptoms, functioning, and HRQOL and aid in understanding the benefit/risk evaluation from the patient's perspective. The following PRO measures will be administered in this study in accordance with the SoA (Table 6), and in the following order:
European Organisation for Research and Treatment of Cancer (EORTC) quality of life questionnaire—core questionnaire (EORTC QLQ-C30)
EORTC quality of life questionnaire—breast cancer module (EORTC QLQ-BR23)
National Eye Institute 25-Item Visual Function Questionnaire (NEI VFQ-25)
EuroQol 5-Dimension 5-level (EQ-5D-5L)
Patient global impression of severity (PGIS)
Patient global impression of change (PGIC) (not applicable at baseline, i.e. Cycle 1 Day 1)
Patient global impression of treatment tolerability (PGI-TT)
Patient global impression of benefit-risk (PGI-BR) (not applicable at baseline, i.e. Cycle 1 Day 1)

Safety Assessments. Planned timepoints for all safety assessments are provided in the SoA (Table 6). Table 9 provides the list of clinical safety laboratory tests to be performed and to the SoA for the timing and frequency.

TABLE 9
Laboratory Safety Variables

| Haematology (whole blood) | Clinical chemistry (serum or plasma) |
|---|---|
| B-Haemoglobin (Hb) | S/P-Creatinine |
| B-Leukocyte count | S/P-Bilirubin, total (TBL) |
| B-Haematocrit | S/P-Conjugated bilirubin |
| B-Red blood cell count | S/P-Unconjugated bilirubin |
| B-Leukocyte differential count (absolute count) | S/P-Alkaline phosphatase (ALP) |
| Neutrophils | S/P-Aspartate transaminase (AST) |
| Lymphocytes | S/P-Alanine transaminase (ALT) |
| Monocytes | S/P-Creatine kinase (CK) |
| Basophils | S/P-Albumin |
| Eosinophils | S/P-Calcium, total |
| B-Platelet count | S/P-Potassium |
| | S/P-Sodium |
| Coagulation parameters | S/P-Glucose |
| activated partial thromboplastin time (aPTT) | S/P-Magnesium |
| international normalised ratio (INR) | S/P-Phosphate |
| | S/P-Urea nitrogen |
| Urinalysis | S/P-Protein, total |
| U-Glucose | S/P-Troponin |
| U-Protein | S/P-NT-proBNP |
| U-Blood | |

Pharmacokinetics. Venous blood samples of approximately 2 mL will be collected from patients receiving AZD9833 for measurement of plasma concentrations of AZD9833 and, if appropriate, metabolite(s) as specified in the SoA (Table 6).

Samples may be collected at additional timepoints or no longer collected during the study if warranted and agreed upon between the Investigator and the Sponsor. Instructions for the collection and handling of biological samples will be provided by the Sponsor or analytical test site. The actual date and time (24-hour clock time) of each sample will be recorded.

Samples collected for analyses of AZD9833 plasma concentration may also be used to evaluate safety or efficacy aspects related to concerns arising during or after the study (i.e., PK samples may be repurposed if required).

Genetics. Patients will be offered the possibility to participate in optional genetic exploratory research. Collection and storing of DNA will be according to each country's local and ethical procedures for future exploratory research into genes/genetic variation that may influence response to treatment. After signing a separate consent for optional genetic research, a 6-mL blood sample will be collected in accordance with the inclusion criteria and SoA (Table 6). If for any reason the blood sample is not drawn on Day 1 according to the SoA, it may be taken at any time up until the last study visit. Although the genotype is a stable parameter, early sample collection is preferred to avoid introducing bias through excluding subjects who may withdraw due to an AE, such subjects would be important to include in any genetic analysis. Only 1 sample should be collected per subject for genetic research during the study.

Biomarkers. Biomarkers will be tested in plasma, blood and tumour samples for the secondary and exploratory objectives to investigate predictive markers of response and/or acquired resistance to AZD9833 and to assess the impact of tumour mutation status on response to treatment and acquired resistance.

The following samples for biomarker research are required and will be collected from all patients in this study as specified in the SoA (Table 6).
Blood and plasma samples for ctDNA
Blood samples for tumour marker CA15-3
Blood samples for CTCs
Archival tumour tissue samples

The following samples for biomarker research are optional and will collected from patients in the study where possible as specified in the SoA (Table 6).
Paired tumour biopsy samples; pre- and on-treatment. Samples will be collected for patients with tumours amenable to biopsies in up to 12 patients per treatment group.
Additional tumour biopsy samples may be collected at time of progression.
   Blood samples for circulating tumour DNA. One 20-mL whole blood sample will be taken at screening, for the isolation of plasma and buffy coat to enable the assessment, analysis and interpretation of circulating tumour DNA. 10-mL blood sample will be taken at all of the other timepoints indicated in the SoA (Table 1) to provide plasma only.

The samples will be used for the extraction and analysis of ctDNA for the analysis of predictive and pharmacodynamic biomarkers to interrogate changes in frequency, level, specific genetic alterations and potential mechanisms of resistance.

Blood samples for tumour marker CA15-3. A blood sample of approximately 2 mL will be taken for cancer antigen CA15-3 assessment at each of the timepoints indicated in the SoA (Table 6). The sample will be analysed at a local laboratory at or near to the Investigator site. Sample tubes and sample sizes may vary depending on laboratory method used and routine practice at the site.

Circulating tumour cells. One 10-mL blood sample will be taken at each of the timepoints as indicated in the SoA (Table 6). These samples will be taken to obtain a preliminary assessment of AZD9833 activity in the tumour by evaluation of pharmacodynamic biomarker changes, which may include but are not limited to total counts, and expression levels of ER, Ki67 protein, and ER regulated genes.

Archival tumour tissue. Formalin-fixed archival tumour tissue embedded in paraffin blocks are to be retrieved for all patients, where available. If baseline biopsy samples can also be collected, retrieval of the archival diagnostic tumour material is still required to provide data on how the tumour has evolved since diagnosis. The archival samples can be derived from the primary tumour and/or metastatic site and, where possible, the most recently acquired archival sample is required.

If this is not possible, a minimum of 20 slides of freshly prepared unstained 5 micron sections from the archival tumour block are accepted, if tumour blocks cannot be submitted. From submitted archival tumour blocks, cores may be removed to construct tissue microarrays for later biomarker analysis. The remaining part of the tumour block may be returned to the institution.

Tumour biopsy in selected patients. Optional paired tumour biopsies will be collected from up to 12 eligible patients in each treatment group. The paired tumour biopsies will be obtained from patients with accessible tumours and who consent to provide biopsy samples. Accessible lesions are defined as tumour lesions that are amenable to repeat biopsy. Paired biopsies of bone tissue are not permitted. The pre-treatment biopsy may be taken during screening as close as possible to starting treatment, ideally no greater than 6 weeks prior to treatment start. The on-treatment biopsy sample may be taken on Day 1 (±7 days) of Cycle 2, but both pre- and on -treatment samples can be taken outside this time window, if agreed with AstraZeneca. The pre-dose and on-treatment biopsies should be taken from the same tumour lesion. Biopsies should be taken within 1 to 12 hours of the last AZD9833 dose where possible. A further tumour biopsy may also be taken on disease progression or at the end of treatment.

The biomarkers to be investigated using tumour samples may include, but will not necessarily be limited to: ER, PgR, Ki67, genomic/genetic alterations, and other ER-regulated gene expression. Where feasible, collection of a tumour biopsy at disease progression is encouraged. This sample will be used to investigate changes in pathway signalling and potential mechanisms of resistance (i.e., genetic alterations or evidence of alternative pathway activation).

Statistical methods. All efficacy analyses will be performed on the full analysis set (FAS). The primary analysis, which is a formal comparison of AZD9833 to fulvestrant, only applies to AZD9833 75 mg and 150 mg treatment arms, and pairwise comparisons only refer to AZD9833 doses 75 mg and 150 mg. Results of all statistical analyses will be presented using 90% confidence intervals (CIs) and 2-sided p-values. The treatment comparisons of interest are each dose level of AZD9833 versus fulvestrant. Data accrued from the AZD9833 300 mg treatment arm will be summarised and reported as appropriate, but will not contribute to the event triggers for the analysis timepoints below.

For the primary analysis, the null hypothesis to be tested is that there is no treatment effect, (i.e., there is no difference in PFS between patients treated with any dose of AZD9833 and patients treated with fulvestrant).
H₀: PFS HR_{AZD9833/fulvestrant}=1
H₁: PFS HR_{AZD9833/fulvestrant}≠1

Each dose of AZD9833 of interest will be compared with fulvestrant in a pairwise comparison. As this is a Phase 2 study, no adjustments for multiplicity will be made. The primary endpoint will be based on the Investigator assessment of progression, as defined by RECIST version 1.1. A sensitivity analysis based on the Blinded Independent Central Review (BICR) of scans will be conducted.

The analysis of the primary endpoint of PFS (as assessed by the Investigator) will occur when recruitment to the study has completed and at least 108 events have occurred for the pairwise comparison of 75 mg and 150 mg AZD9833 doses versus fulvestrant (approximately 75% maturity). A further analysis of PFS may also be conducted at a later timepoint based on more mature data, particularly in the subgroups of interest.

A sample size of approximately 288 patients, randomised in equal proportions to the 4 treatment groups will be required to observe a total of at least 108 PFS events for each pairwise comparison against fulvestrant. A HR of 0.59 for each pairwise treatment comparison versus fulvestrant is of interest. Under the assumption that a 5-month median PFS will be observed on fulvestrant, this is equivalent to a 3.5-month increase in median PFS over fulvestrant. A minimum of 108 events for the pairwise comparison of each AZD9833 dose of interest versus fulvestrant will provide 86% power at the 2-sided 10% significance level if the assumed true treatment effect is HR=0.59.

The objective response rate (ORR) will be compared between AZD9833 (each dose level) and fulvestrant using a logistic regression model adjusting for prior use of CDK4/6 inhibitors and presence of lung and/or liver metastases.

The change from baseline in tumour size at 16 weeks and best change from baseline in tumour size will also be summarised and presented by randomised treatment group.

The overall survival (OS) data will be analysed at the time of the primary analysis of PFS and will use the same methodology and model (provided there are enough events available for a meaningful analysis). Further survival analyses may be conducted after 50% and 75% of patients have died, or other maturities as may be appropriate.

The CBR24 will be summarised by treatment group and analysed using a logistic regression model (similarly to the analysis for the ORR).

All safety analyses will be performed on the safety analysis set. Safety data will not be analysed formally.

Populations for analyses. For purposes of analysis, the following populations are defined in Table 10.

TABLE 10
Study Populations

| Population | Description |
|---|---|
| Enrolled | All patients who sign the ICF. |
| Full analysis | All randomised patients, with treatment groups assigned in accordance with the |
| set (FAS) | randomisation, regardless of the treatment actually received. |
| | Patients who are randomised but do not subsequently receive treatment will be |
| | included in the FAS. The analysis of data using the FAS therefore follows the |
| | principles of ITT. |
| Safety | All patients who received any amount of study treatment (AZD9833 or fulvestrant), |
| analysis set | regardless of whether that was the randomised therapy intended or whether they |
| | received therapy without being randomised and for whom any post-dose data are |
| | available. |
| | Patients randomised to AZD9833 who received only fulvestrant will be accounted |
| | for in the fulvestrant treatment group. |
| | Safety data will not be formally analysed but summarised using the safety analysis |
| | set, according to the treatment/AZD9833 dose level actually received. |
| Pharmacokinetics (PK) | All patients who receive at least 1 dose of AZD9833 per the protocol, for whom |
| analysis set | there is at least 1 reportable PK concentration. |
| Pharmacodynamic | All patients who received at least 1 dose of AZD9833 or fulvestrant per the |
| analysis set | protocol, and who: |
| | had evaluable paired tumour samples by central pathology assessment |
| | had no major protocol deviations that impacted the biomarkers analysis. |

Statistical analyses. All Sponsor personnel involved in the study will remain blinded to the aggregate efficacy data until after database lock for the primary analysis. Following database lock for the primary analysis, all study team members will become unblinded.

Efficacy analyses. All efficacy analyses will be performed on the FAS. The primary analysis, which is a formal comparison of AZD9833 to fulvestrant, only applies to AZD9833 75 mg and 150 mg treatment arms and pairwise comparisons only refer to AZD9833 doses 75 mg and 150 mg. Results of all statistical analyses will be presented using 90% CIs and 2-sided p-values. The treatment comparison of interest is each dose level of AZD9833 versus fulvestrant. Data accrued from the AZD9833 300 mg treatment arm will be summarised and reported as appropriate.

Investigator's assessment of tumour response per RECIST version 1.1. From the Investigators review of the imaging scans, the measurement of TLs (where appropriate), assessment of NTLs and new lesions will be used to determine the overall visit response of each patient according to RECIST version 1.1: patients will be programmatically assigned an overall visit response of complete response (CR), partial response (PR), stable disease (SD), or progressive disease (PD) depending on the status of their disease compared with baseline and previous assessments. The efficacy endpoints of PFS, CBR24, ORR and DoR will be derived programmatically from the overall visit responses determined for each visit. The endpoint of percentage change in sum of TLs will be derived programmatically from the TL tumour measurements.

Blinded independent central review of tumour response. A sensitivity analysis for the primary endpoint for this study will be based on the BICR of the radiological scans. For each patient, the BICR will define the overall visit response (i.e., the response obtained overall at each visit by assessing TLS, NTLs and new lesions) data and no programmatic derivation of visit response is necessary. PFS will be derived programmatically from the overall visit responses determined for each visit. A BICR of all patients will be performed for the final database lock for the primary analysis of PFS. The BICR will cover all available scans up to the respective DCO.

Primary endpoint: progression-free survival. The analysis of the primary endpoint of PFS (as assessed by the Investigator) will occur when recruitment to the study has completed and at least 108 events have occurred for the pairwise comparison of each AZD9833 dose versus fulvestrant (approximately 75% maturity). A further analysis of PFS may also be conducted at a later timepoint based on more mature data, particularly in the subgroups of interest.

The PFS is defined as the time from date of randomisation to date of objective disease progression or death (by any cause in the absence of progression), regardless of whether the patient withdraws from randomised therapy or receives another anti-cancer therapy prior to progression (i.e., date of PFS event or censoring−date of randomisation+1).

Patients who have not progressed or died at the time of analysis will be censored at the time of the latest date of assessment from their last evaluable assessment (per RECIST version 1.1). However, if the patient progresses or dies immediately after 2 or more consecutive missed visits, the patient will be censored at the time of the latest evaluable assessment (per RECIST version 1.1) prior to the 2 missed visits. The PFS time will always be derived based on scan/assessment dates and not on visit dates.

PFS will be analysed based on the FAS using a Cox proportional hazards model, allowing for the effect of treatment and including terms for the stratification factors; prior use of CDK4/6 inhibitors (yes/no) and presence of lung and/or liver metastases (yes/no). A stratified log-rank test adjusting for prior use of CDK4/6 inhibitors and presence of lung and/or liver metastases will be used to compare all AZD9833 doses against fulvestrant.

The stratification variables in the statistical modelling will be based on the values entered into the IWRS at randomisation, even if it is subsequently discovered that these values were incorrect. If considered necessary, a sensitivity analysis may be conducted based on the correct assignment.

The HRs for each treatment comparison against fulvestrant will be estimated along with their 90% CIs and 2-sided p-values.

Kaplan-Meier plots of PFS will be presented by treatment arm. Summaries of the number and percentage of patients experiencing a PFS event, and the type of event (progression or death) will be provided along with the median PFS, and the proportion of patients progression-free at 6 months and 1 year for each treatment arm.

The assumption of proportionality will be assessed, should evidence of non-proportionality exist a time-dependent covariate will be fitted to assess the extent to which this represents random variation. Full details will be provided in the SAP.

As a sensitivity analysis to the primary endpoint, the above analysis methods will be repeated based on the BICR.

Collected RECIST version 1.1 data (Investigator determined and BICR) will be listed for all randomised patients.

Subgroup analyses. The primary endpoint will also be summarised and analysed for the subgroups of patients with prior use of CDK4/6 inhibitors (yes/no), provided there are enough events available for a meaningful analysis; if not, descriptive summaries will be provided. Further subgroups of interest may be defined based on patient characteristics at baseline, these will be illustrated in a forest plot comparing the HR and 90% CIs. The subgroups of interest, along with any sensitivity analyses will be fully defined in the SAP.

Secondary endpoints: objective response rate. The ORR is defined as the percentage of patients with at least 1 Investigator-assessed visit response of CR or PR prior to any evidence of progression. The ORR will be based on a subset of the FAS with measurable disease at baseline. A response does not need to be confirmed to be included in the calculation of ORR.

The ORR will be compared between AZD9833 (each dose level) and fulvestrant using a logistic regression model adjusting for prior use of CDK4/6 inhibitors and presence of lung and/or liver metastases. The results of the analysis will be presented in terms of an odds ratio for each treatment comparison together with its associated profile likelihood 90% CI and 2 sided p values (based on twice the change in log-likelihood resulting from the addition of a treatment factor to the model).

Summaries will be produced that present the number and percentage of patients with a tumour response (CR/PR), these will include frequencies of confirmed complete responses and partial responses, in addition to unconfirmed complete and partial responses, stable disease, progressive disease and not evaluable.

Secondary endpoints: duration of response. The DoR will be defined as the time from the date of first documented response until date of documented progression or death in the absence of disease progression. The time of the initial response will be defined as the latest of the dates contributing towards the first visit response of PR or CR. If a patient does not progress or die following a response, then their DoR will use the PFS censoring time. Descriptive data will be provided for the duration of response in responding patients, including the associated Kaplan-Meier curves.

Secondary endpoints: change in tumour size. One of the secondary outcome variables for this study is percentage change from baseline in the sum of TL diameters at 16 weeks. The percentage change in tumour size at 16 weeks will be obtained for each patient, based on RECIST measurements taken at baseline and at 16 weeks. Tumour size is the sum of the longest diameters of the TLs. TLs are measurable tumour lesions. Baseline for RECIST is defined to be the last evaluable assessment prior to randomisation.

Secondary endpoints: best percentage change. The absolute change and percentage change from baseline in the sum of tumour size at each visit will be calculated. The best change in tumour size (i.e., depth of response) is the largest decrease from baseline or the smallest increase from baseline in the absence of a reduction and will include all assessments prior to the earliest of death in the absence of progression, any evidence of progression, the start of subsequent anti-cancer therapy or the last evaluable RECIST assessment if the patient has not died, progressed or started subsequent anti-cancer therapy.

The absolute value, the change in tumour size from baseline and the percentage change in tumour size from baseline will be summarised using descriptive statistics and presented at each timepoint and by randomised treatment group. The change from baseline in tumour size at 16 weeks and best change from baseline in tumour size will also be summarised and presented by randomised treatment group. Normality of the data will be assessed and if appropriate the effect of AZD9833 on percentage change in tumour size will be estimated from an analysis of covariance (ANCOVA) model. The number of patients, unadjusted mean and least squares means (LSmeans) for each treatment group will be presented, together with the difference in LSmeans, 90% CI and corresponding 2-sided p-values. If the assumptions do not hold, an appropriate transformation of the data will be investigated, full details of which will be provided in the SAP. Tumour size will also be presented graphically using waterfall plots and spider plots split by treatment as appropriate.

Secondary endpoints: overall survival. The OS is defined as the time from the date of randomisation until death due to any cause regardless of whether the patient withdraws from randomised therapy or receives another anti-cancer therapy. Any patient not known to have died at the time of analysis will be censored based on the last recorded date on which the patient was known to be alive.

Note: Survival follow-up phone calls will be made in the week following the date of DCO for the analysis, and if patients are confirmed to be alive or if the death date is after the DCO date these patients will be censored at the date of DCO.

The OS data will be analysed at the time of the primary analysis of PFS and will use the same methodology and model (provided there are enough events available for a meaningful analysis [>20% maturity in OS], if not, descriptive summaries will be provided). Further survival analyses may be conducted after 50% and 75% of patients have died.

Secondary endpoints: clinical benefit rate at 24 weeks. The CBR24 is defined as the percentage of patients who have a best objective response (BoR) of CR or PR in the first 25 weeks (to allow for a late assessment within the assessment window) or who have SD (without subsequent cancer therapy) for at least 23 weeks after start of treatment (to allow for an early assessment within the assessment window). The CBR will be defined based on the Investigator's assessment of RECIST, and will be summarised by treatment group and analysed using a logistic regression model (similarly to the analysis for the ORR).

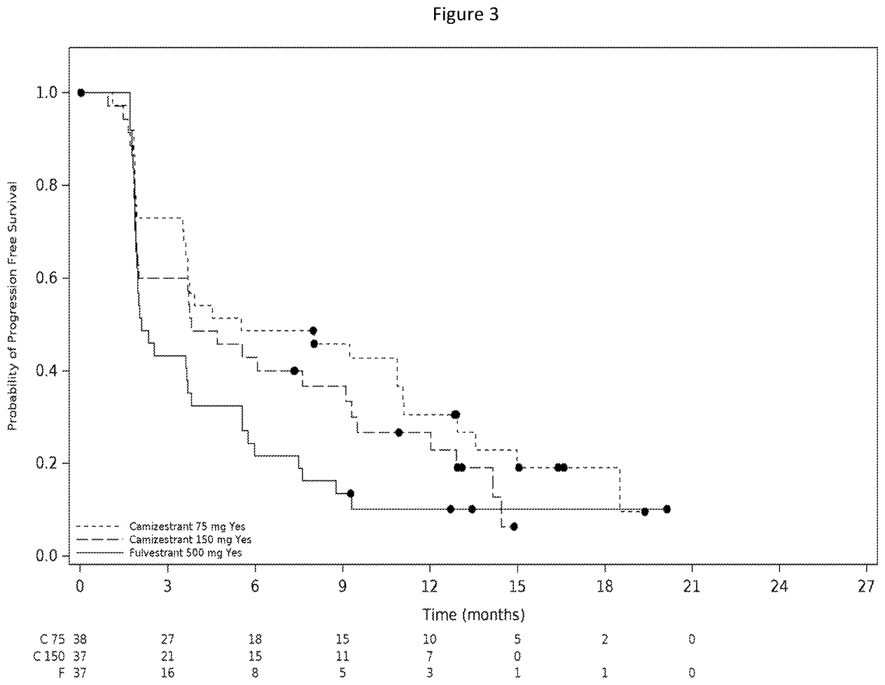

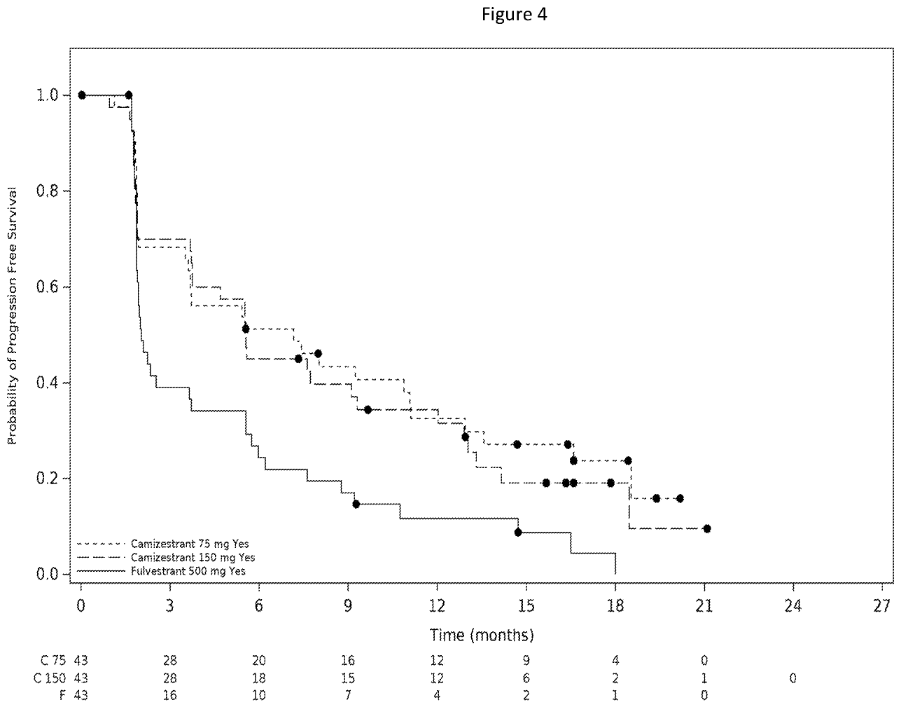

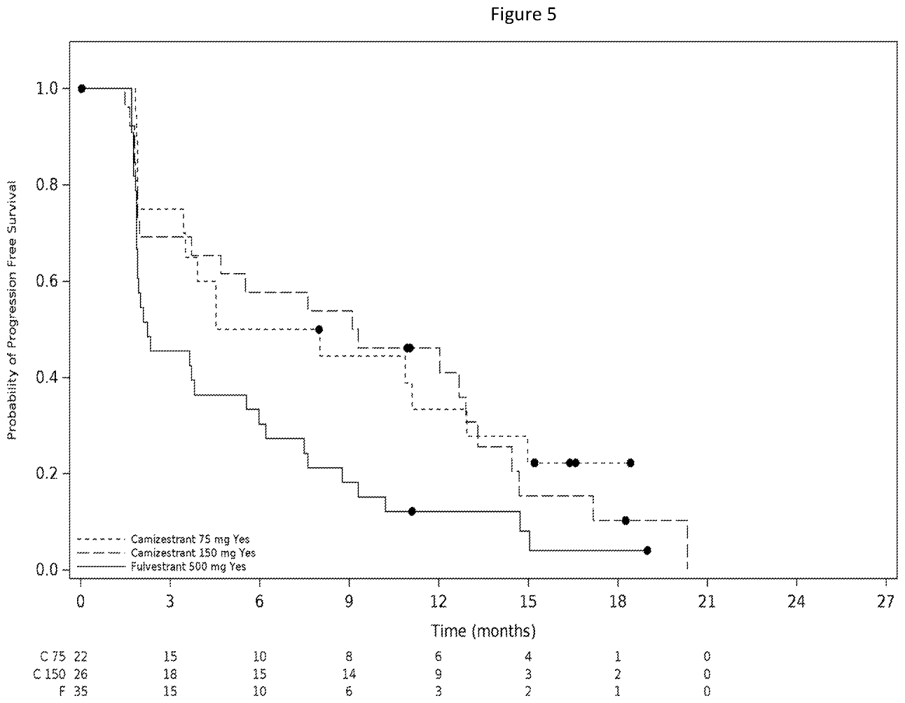

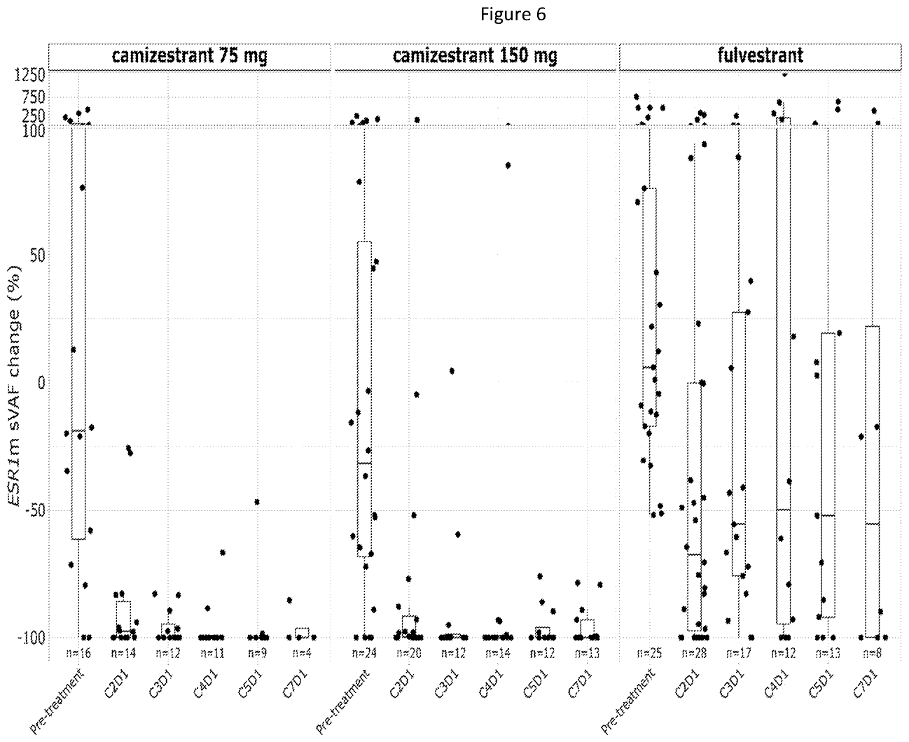

Results obtained from the described clinical trial protocol are presented in FIGS. 1 to 6.

1. A method of treatment of HR+, HER2−, metastatic or loco-regionally recurrent breast cancer, comprising administering a therapeutically effective amount of camizestrant or a pharmaceutically acceptable salt thereof to a patient in need thereof wherein the cancer has recurred or progressed following at least one prior line of endocrine therapy; and wherein the camizestrant or a pharmaceutically acceptable salt thereof is orally administered once daily at a dose of 75 mg.

2. The method of treatment according to claim 1 wherein:
a) the median time to disease progression is at least 3.5 months more than that observed with fulvestrant treatment; and/or
b) the hazard ratio for ngSERD treatment relative to fulvestrant treatment is less than or equal to 0.67; and/or
c) the median time to disease progression is at least 7 months.

3. The method of treatment according to claim 1, wherein the cancer has recurred or progressed following at least one prior line of therapy with a CDK4/6 inhibitor.

4. The method of treatment according to claim 3 wherein:
a) the median time to disease progression is at least 1.7 months more than that observed with fulvestrant treatment; and/or
b) the hazard ratio for ngSERD treatment relative to fulvestrant treatment is less than or equal to 0.68; and/or
c) the median time to disease progression is at least 3.8 months.

5. The method of treatment according to claim 1, wherein the cancer has been identified as having visceral metastases.

6. The method of treatment according to claim 5 wherein:
a) the median time to disease progression is at least 3.6 months more than that observed with fulvestrant treatment; and/or
b) the hazard ratio for ngSERD treatment relative to fulvestrant treatment is less than or equal to 0.55; and/or
c) the median time to disease progression is at least 5.6 months.

7. The method of treatment according to claim 1, wherein the cancer has been identified as having a mutation of the estrogen receptor α.

8. The method of treatment according to claim 7, wherein the cancer has been identified as having a mutation to the estrogen receptor a selected from E380Q, V422del, S463P, L536H, L536P, L536R, Y537C, Y537D, Y537N, Y537S and D538G.

9. The method of treatment according to claim 7, wherein the identification of a mutation of the estrogen receptor a is made on the basis of test of a sample obtained from the patient.

10. The method of treatment according to claim 9, wherein the sample obtained from the patient is a tumour biopsy or blood sample.

11. The method of treatment according to claim 10, wherein the identification of a mutation of the estrogen receptor a is performed by analysing circulating tumor DNA.

12. The method of treatment according to claim 1, wherein:
a) the median time to disease progression is at least 4 months more than that observed with fulvestrant treatment; and/or
b) the hazard ratio for ngSERD treatment relative to fulvestrant treatment is less than or equal to 0.55; and/or
c) the median time to disease progression is at least 6.3 months.

13. (canceled)

14. (canceled)

15. (canceled)

16. The method of treatment according to claim 1, wherein the method improves overall survival relative to treatment with fulvestrant.

17. The method of treatment according to claim 1, wherein the method delivers an improvement in the clinical benefit rate at 24 weeks relative to fulvestrant.

18. The method of treatment according to claim 1, wherein the method delivers an improvement in the objective response rate relative to fulvestrant.

19. A method of treatment of HR+, HER2−, metastatic or loco-regionally recurrent breast cancer, comprising administering a therapeutically effective amount of camizestrant or a pharmaceutically acceptable salt thereof to a patient in need thereof, wherein the cancer has recurred or progressed following at least one prior line of endocrine therapy, and wherein:
a) the method results in an improvement in the median time to disease progression of at least 3.5 months relative to that observed with fulvestrant treatment; and/or
b) the method results in a hazard ratio for camizestrant treatment relative to fulvestrant treatment of less than or equal to 0.67; and/or
c) the method results in a median time to disease progression of at least 7 months.

20. (canceled)

21. A method of treatment of HR+, human epidermal growth factor receptor 2 negative HER2−, metastatic or loco-regionally recurrent breast cancer, comprising administering a therapeutically effective amount of a pharmaceutical composition comprising camizestrant or a pharmaceutically acceptable salt thereof to a patient in need thereof, wherein the cancer has recurred or progressed following at least one prior line of endocrine therapy, and wherein:
a) the method results in an improvement in the median time to disease progression of at least 3.5 months relative to that observed with fulvestrant treatment; and/or
b) the method results in a hazard ratio for camizestrant treatment relative to fulvestrant treatment of less than or equal to 0.67; and/or
c) the method result in a median time to disease progression of at least 7 months.

22. (canceled)
